(19) United States
(12) Patent Application Publication
(10) Pub. No.: US 2012/0213796 A1
(43) Pub. Date: Aug. 23, 2012

(54) POLYCLONAL ANTIBODY COMPOSITIONS

(76) Inventors: Barbara S. Fox, Wayland, MA (US); Eileen F. Bostwick, Waltham, MA (US); Michael S. Quesenberry, Douglas, MA (US)

(21) Appl. No.: 13/402,527

(22) Filed: Feb. 22, 2012

Related U.S. Application Data
(60) Provisional application No. 61/445,201, filed on Feb. 22, 2011.

(51) Int. Cl. A61K 39/395; C07K 16/24; A61P 1/00; C07K 16/18

(52) U.S. Cl. 424/158.1; 530/387.1; 424/130.1; 530/389.2

(57) ABSTRACT

The present invention provides purified immunoglobulin compositions derived from the colostrum of a bovine immunized with a target antigen. The immunoglobulin composition comprises polyclonal antibodies specific for the target antigen and is depleted of non-immunoglobulin factors. The invention includes methods of manufacturing the compositions of the invention. The invention further includes pharmaceutical formulations comprising a purified immunoglobulin composition of the invention and an optional pharmaceutically acceptable excipient.

### RELATED APPLICATION

This application claims the benefit of U.S. Provisional Application No. 61/445,201, filed on Feb. 22, 2011. The entire teaching of the above application is incorporated herein by reference.

### GOVERNMENT SUPPORT

The invention was supported, in whole, or in part, by NIH grant numbers 1R43DE019735-01 and 1R43DK083810-01A1 and by HHS contract HHS0100201100027C. The Government has certain rights in the invention.

### BACKGROUND OF THE INVENTION

Antibodies are an important class of pharmaceuticals. Antibodies specific for a target antigen have proven to be highly effective therapeutics in treating cancers and autoimmune disease, and their use has been of great benefit to afflicted patients. Antibodies are generally highly specific for a particular target and thus tend to have less off-target toxicity than is seen with small molecule therapeutics.

WO 2009/046168; WO 2009/020748 and US 20070184049 A1 describe the use of polyclonal antibodies derived from the milk of immunized mammals for use as therapeutics topically delivered to the digestive tract to target antigens that modulate the pathogenesis of one or more diseases. Colostrum and milk, particularly from bovine sources, are a uniquely safe source of polyclonal antibody for oral delivery to a human patient. There is already extensive human exposure to bovine immunoglobulin, as regular milk contains approximately 1.5 g/L IgG. However, milk and colostrum contain other components which on their own have therapeutic uses, but that may not be ideal in the context of treating certain diseases using polyclonal antibodies derived from a milk source. In addition to specific antibodies induced by immunization of the donor animal, milk and colostrum contain antibody with other specificities and many other biologically active non-immunoglobulin factors including, but not limited to proteins, peptides, and small molecules (reviewed in Korhnonen {Korhonen and Pihlanto, 2007, Curr Pharm Des, 13, 829-43} and Liang {Liang et al., 2011, Int J Environ Res Public Health, 8, 3764-76}).

Specific non-immunoglobulin components in milk and colostrum, many of which have biological activity either alone or in combination include lactoferrin, lactoperoxidase, alpha-lactalbumin, beta-lactoglobulin, transferrin, lysozyme, EGF, FGF, IGF-1, IGF-2, TGF-α, TGF-β1, TGF-β2, PDGF, VEGF, NGF, CTGF, Growth Hormone, Insulin, protease, PRP, glutamine, polyamines, nucleotides, prolactin, somatostatin, oxytocin, luteinizing hormone-releasing hormone, TSH, thyroxine, calcitonin, estrogen, progesterone, IL-1b, TNF, IL-6, IL-10, IL-8, G-CSF, Ifn-gamma, GM-CSF, C3, C4, mammary-derived growth factor II, human milk growth factor III; growth hormone and growth hormone releasing factor, casein, casein-derived peptides, Vitamins B1, B2, B6, B12, E, A, C, Folic Acid, pantothenic acid, beta-carotene, glycogen, retinoic acid, calcium, chromium, iron, magnesium, phosphorous, potassium, sodium, zinc, isoleucine, leucine, histidine, methionine, lysine, threonine, phenylalanine, valine, tryptophan, arginine, cysteine, glutamic acid, alanine, tyrosine, proline, aspartic acid, serine, β-2 microglobulin, haemopexin, haptoglobulin, orotic acid, peroxidase, and xanthine oxidase.

Colostrum is widely used as a nutritional supplement and has been studied as a therapeutic. {Khan et al., 2002, Aliment Pharmacol Ther, 16, 1917-22}. It has also been shown to be effective in animal models of colitis {Bodammer et al., 2011, J Nutr, 141, 1056-61}.

Many researchers have taken advantage of the therapeutic uses of such non-immunoglobulin components of colostrum and milk by concentrating one or more of the above-listed non-immunoglobulin components and depleting out other components such as immunoglobulin and casein. Potential therapeutic uses for such concentrated growth factors include the treatment of digestive ailments and the treatment of digestive inflammation. Colostrum has been considered as a beneficial treatment for a variety of intestinal ailments. Growth factors derived from milk or colostrum have been considered for their use in the chemotherapy-induced mucositis. Methods for enriching for milk-derived growth factors and other bioactive components are known in the art. The art discloses compositions of bovine derived antibodies for oral administration of the treatment of diseases, particularly gastrointestinal diseases resulting from infection by a pathogen. However, the art does not contemplate the use of such antibodies in isolation from non-immunoglobulin components found in milk or colostrum, particularly when delivered orally. Indeed it was previously believed that such non-immunoglobulin components of colostrum stabilize the antibodies for oral administration.

It has not previously been appreciated that the presence of multiple active non-immunoglobulin factors in a pharmaceutical antibody product may be problematic. Some of the issues raised by the presence of non-immunoglobulin bioactives are listed here.

First, levels of some of these non-immunoglobulin factors are affected by the health of the cow, by farm management practices, and by the stage of lactation during which collection occurred. For instance, in one survey of colostrum from 55 cows, {Kehoe et al., 2007, J Dairy Sci, 90, 4108-16} the average level of lactoferrin was 0.8 mg/ml but the range from individual cows was 0.1 mg/ml-2.2 mg/ml. This introduces a source of variability into the product which may make it difficult to achieve the consistency of manufacture required for a licensed biologic.

The variability in expression of these non-immunoglobulin factors is particularly challenging because it has not been possible to cleanly identify a single component or mixture of components that is responsible for the biological activity of colostrum. On the one hand, this makes it very difficult to achieve product uniformity. On the other hand, it makes it difficult to set specifications around the product.

Second, some of these non-immunoglobulin factors may act on the same pathways or disease processes that are being targeted by the specific antibodies in the therapeutic. This will make it difficult to evaluate the therapeutic benefit that results from administration of the specific antibody.

Third, some of these non-immunoglobulin factors may be associated with safety concerns, particularly when given to patients with gastrointestinal diseases. This is particularly true when the antibody product is intended to be administered chronically. For example, long-term exposure to growth factors may increase the risk of malignancy.

Thus there is a need to develop compositions and methods to permit the manufacture of a consistent antibody product that is free from potentially therapeutically confounding activities including the presence of non-immunoglobulin factor impurities.

### SUMMARY OF THE INVENTION

The present invention provides compositions derived from a biological source wherein the composition comprises polyclonal antibodies that are specific for a target antigen. In one embodiment, the composition is a purified and isolated immunoglobulin composition that is depleted of non-immunoglobulin factors. In one embodiment, the biological source is milk or colostrum. In one preferred embodiment the biological source is milk or colostrum from an animal immunized with the target antigen or immunogenic portion thereof. In one embodiment, the compositions are depleted of lactoferrin. In one embodiment, the compositions are depleted of low molecular weight growth factors. In one embodiment, the compositions are depleted of non-immunoglobulin factors and are further depleted of immunoglobulins that are not specific for the target antigen. The invention includes methods of manufacturing the compositions of the invention. The invention further includes pharmaceutical compositions in accordance with the invention and methods of using such compositions for the treatment of diseases in a patient wherein such diseases are modulated by the activity of the target antigen in the patient.

### BRIEF DESCRIPTION OF THE FIGURES

FIG. 1 is a reducing SDS PAGE analysis of colostrum samples processed under different conditions as discussed in Example 5.

FIG. 2 is a reducing SDS-PAGE analysis of samples from the MEP bind and pH elute of the Capto-S flow through material.

FIG. 3: Size-Exclusion Chromatography Analysis of the MEP Eluate.

FIG. 4: Reducing SDS PAGE analysis of pilot scale preparation on MEP resin.

FIG. 5: Reducing SDS-PAGE analysis comparing MEP and Capto-S.

FIG. 6: Reducing SDS PAGE analysis comparing different neutralization techniques.

FIG. 7: Reducing SDS PAGE analysis of samples from sequential flow through chromatography.

FIG. 8: Reducing SDS PAGE analysis of samples purified on serial Capto-S/Capto-Q column.

FIG. 9: Densitometric analysis of reducing SDS PAGE analysis of samples purified on serial Capto-S/Capto-Q column.

FIG. 10: Identification of gel fragments from reducing SDS PAGE excised for mass spectrometry analysis.

FIG. 11: Reducing SDS PAGE analysis of polyclonal antibody compositions purified under different conditions.

FIG. 12: Densitometric analysis of reducing SDS PAGE analysis of polyclonal antibody compositions purified under different conditions.

FIG. 13: ELISA analysis of affinity purified anti-gliadin antibody binding to gliadin (A) or to peptide 56-89 (SEQ ID NO: 1) (B).

FIG. 14: Reducing SDS-PAGE (A) and densitometric analysis of composition purified with Capto-S/Capto-Q followed by ultrafiltration.

### DETAILED DESCRIPTION OF THE INVENTION

The term “immunoglobulin(s) (Ig) as used herein refer to a polypeptide comprising a framework region from an immunoglobulin gene or fragments thereof that specifically binds and recognizes an antigen. Immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD (not found in bovines) and IgE, respectively. Typically, the antigen-binding region of an immunoglobulin will be most critical in specificity and affinity of binding to a target receptor. An exemplary immunoglobulin structural unit comprises a tetramer and is also referred to herein as an “antibody” or “antibodies” and include polyclonal antibodies. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Immunoglobulins exist, e.g., as intact antibodies or as a number of well-characterized antibody fragments produced by degradation with various peptidases. (e.g. Fab, F(ab′)₂, Fab′, Fc). Immunoglobulin(s) also exist, for example, as fragments that may be present in a biological source such as milk or colostrum that are the result of natural degradation or degradation associated with processing of the milk or colostrum. As used herein the term immunoglobulin(s) includes polypeptides that are associated with immunoglobulins such as the secretory component and J chain components associated with IgA and IgM. Therefore, as used herein the term immunoglobulin (Ig) compositions refers to compositions of intact antibodies (including polyclonal antibodies) or fragments thereof or protein components associated therewith derived from all immunoglobulin isotypes.

The terms “polyclonal antibody” and “polyclonal antibodies” as used herein refer to a composition of different antibody molecules which is capable of binding to or reacting with several different specific antigenic determinants on the same or on different antigens. Polyclonal antibody preparations isolated from the blood, milk, colostrum or eggs of immunized animals typically include antibodies that are not specific for the target antigen in addition to antibodies specific for the target antigen. Thus, the term “polyclonal antibody” as used herein refers both to antibody preparations in which the antibody specific for the target receptor has been enriched and to preparations that are not so enriched. Preferably, polyclonal antibodies are prepared by immunization of an animal with the target antigen or portions thereof as specified below.

The term “non-immunoglobulin factors” as used herein includes non-immunoglobulin proteins and peptides, non-immunoglobulin macromolecules and small molecules. Antibodies that are present in the biological source such as colostrum, milk or serum that are not specific for the target antigen are referred to herein as “non-specific antibodies”. The term “target antigen” refers to the antigen to which the polyclonal antibodies of a composition are intended to bind.

In one embodiment, the polyclonal antibodies of a composition of the invention are specific for an endogenous target antigen. An “endogenous target antigen” is an antigen that is manufactured by cells or tissues of the human or animal patient being treated with the polyclonal antibodies of the invention. Antigens synthesized by organisms resident within the body of the patient including non-infectious, “friendly” bacteria or infectious pathogenic agents (e.g. viruses, bacteria, fungi, protozoa and parasites) are not considered endogenous target antigens in accordance with this invention. In one embodiment, the antibodies of the invention are specific for exogenous agents, where “exogenous agents” are defined as those agents that are not endogenous target antigens. Agents that are synthesized by microorganisms resident in the body of the animal being treated with the antibodies are exogenous agents. In one embodiment of the invention, antibodies are not targeted to infectious agents, including viruses, bacteria, fungi, protozoa and parasites. In one embodiment of the invention, target antigens do not include the cytotoxic or immunogenic components of viruses, bacteria, fungi, protozoa and parasites.

A “biological source” refers to the source from which the compositions of the invention comprising polyclonal antibodies are derived wherein such source comprises at least one biological component including but not limited to cells, cell components, tissue, serum, milk and colostrum.

In a preferred embodiment, the biological source for the compositions of the invention comprising polyclonal antibodies is milk or colostrum. In one preferred embodiment the milk or colostrum is derived from an animal that has been immunized with the target antigen or immunogenic portion thereof. The “immunogenic portion” of an antigen is any portion of the antigen that is capable of inducing an immune response in the host animal being immunized with the antigen and that preferably causes the animal to raise polyclonal antibodies against the target antigen.

As is understood in the art, the target antigen is an antigen that is present in a patient who will ultimately be treated with the polyclonal antibody compositions of the invention that are specific to the target antigen. As such the polyclonal antibodies in accordance with the invention will bind the target antigen when administered to the patient. For example, for a polyclonal antibody specific for TNF, the target antigen is preferably human TNF-alpha (TNF) when the patient is a human patient.

In a preferred embodiment, a composition comprising the polyclonal antibodies specific for a target antigen is isolated from the milk or colostrum of a bovine, preferably an immunized cow. In one preferred embodiment the polyclonal antibodies are bovine IgG antibodies. In a particularly preferred embodiment, the polyclonal antibodies are bovine antibodies of mixed Ig isotypes present in milk or colostrum including IgA, IgM and IgG.

Bovine colostrum (early milk) is a preferred source of polyclonal antibody compositions for this invention. In cows, antibody does not cross the placenta, and thus all passive immunity is transferred to the newborn calf through the colostrum. As a result, cows secrete a large bolus of antibody into the colostrum immediately after parturition and approximately 50% of the protein in colostrum is immunoglobulin. In the first 4 hours after birth, immunoglobulin concentrations of 50 mg/ml are typically found in the colostrum, dropping to 25-30 mg/ml 24 hours later. As used herein the term ‘colostrum’ refers to the lacteal secretions produced by the cow within the first 3 to 4 days after parturition. In some instances it will be specified that colostrum is isolated from a particular time frame after parturition (e.g. first milking colostrum, first day colostrum or colostrum from the first 3 to 4 days after parturition).

Colostrum and milk are a uniquely safe source of polyclonal antibodies for oral delivery because there is already extensive human exposure to bovine immunoglobulin as regular milk contains up to 1.5 g/L IgG.

Methods of production of polyclonal antibodies in an animal are known to those of skill in the art. An appropriate animal is immunized with all or a portion of a target antigen using a standard adjuvant, such as Freund's adjuvant, and a standard immunization protocol. For isolation of antibody in colostrum, the immunizations are timed such that specific antibody levels will be at the desired level at the time of parturition. The animal's immune response to the immunogenic preparation may be monitored by taking test bleeds and determining the titer of reactivity to target receptor. When measurably high titers of antibody to the immunogen are obtained, colostrum, milk or serum is collected from the animal and a composition comprising antibodies are obtained. Further fractionation of the antibody composition to enrich for antibodies reactive to the target antigen may be carried out.

In addition to polyclonal antibodies specific to a target antigen induced by immunization of the donor animal, milk and colostrum contain antibody with other specificities (referred to here as “non-specific immunoglobulins”) and many other proteins, peptides, and small molecules (referred to here as “non-immunoglobulin factors”). These non-immunoglobulin factors have a variety of biological activities and have generally been thought to be either benign or beneficial.

In one aspect of this invention, non-immunoglobulin factors are depleted from polyclonal antibody compositions of the invention during the manufacturing process. This depletion may be done by absorption of the impurities or the immunoglobulin on to affinity columns. Alternatively, this depletion can be performed using size exclusion chromatography or similar techniques. Alternatively, this depletion can be performed using ultrafiltration/diafiltration or similar techniques. Alternatively, this depletion can be performed by absorption of the impurities or the immunoglobulin on to ion exchange columns. A combination of the above-described methods for purifying and isolating immunoglobulins in accordance with the invention may be used.

In one aspect of this invention, the levels of specific non-immunoglobulin factors are monitored during in-process testing and as part of release testing of compositions comprising polyclonal antibodies directed to specific target antigens. In one embodiment, levels of all non-immunoglobulin growth factors are reduced at least 5 fold below the average levels in colostrum. In one embodiment, levels of all non-immunoglobulin growth factors are reduced at least 10 fold below the average levels in colostrum. In one embodiment, the polyclonal compositions of the invention are substantially free of non-immunoglobulin factors.

In one preferred embodiment, the non-immunoglobulin factor depleted from polyclonal antibody compositions of the invention is lactoferrin. In one preferred embodiment, the non-immunoglobulin factors depleted from polyclonal antibody compositions of the invention are one or more specific growth factors. In one embodiment, one or more specific growth factors are depleted at least 10-fold below their natural levels in colostrum and preferably compositions of the invention are substantially free of growth factors.

Growth factors include but are not limited to insulin-like growth factor-1 (IGF-1), insulin-like growth factor-2 (IGF-2), epidermal growth factor (EGF), nerve growth factor (NGF), fibroblast growth factor (FGF), transforming growth factor-alpha (TGF-α), transforming growth factor-beta (TGF-β), platelet-derived growth factor (PDGF), vascular endothelial growth factor (VEGF), connective tissue growth factor (CTGF), growth hormone and insulin.

Table 1 provides data showing general levels of various non-immunoglobulin factors naturally found in milk and colostrum (Ontsouka et al., J. Dairy Sci. 86:2005-2011).

TABLE 1

| Factor | Colostrum (day 2) | Milk |
|---|---|---|
| IGF-1 (ug/ml) | 103 ± 21 | 4 ± 1 |
| Insulin (ug/ml) | 4.55 ± 1.04 | 0.37 ± 0.02 |
| Prolactin (ug/ml) | 120 ± 16 | 15.4 ± 1.0 |
| TNF-alpha (ug/ml) | 5.0 ± 0.6 | 1.8 ± 0.2 |
| Gamma- | 137 ± 9 | 24 ± 8 |
| glutamyltransferase | | |
| (ukat/L) | | |

Table 2 provides additional data showing general levels of various non-immunoglobulin factors naturally found in milk and colostrum (Su, C. K., and B. H. Chiang (2003) J Dairy Sci., 86:1639-1645).

TABLE 2

| Factor | Colostrum | Milk |
|---|---|---|
| Lactoferrin (mg/ml) | 1.0 | Negligible |
| BSA (mg/ml) | 1.0 | 0.4 |
| Beta-lactoglobulin (mg/ml) | 6.0 | 3.2 |
| Alpha-lactalbumin | 1.1 | 1.1 |

Table 3 provides data showing normal levels of various non-immunoglobulin factors found in milk and colostrum (Playford et al., 2000, Am. J. Clin. Nutr. 72:5-14).

TABLE 3

| Factor | Colostrum | Milk |
|---|---|---|
| TGF-beta (ug/ml) | 20-40 | 1-2 |
| IGF-1 (ug/ml) | 0.5 | 0.01 |

Non-immunoglobulin factors including growth factors that may be depleted from polyclonal antibody compositions of the invention derived from milk or colostrum in accordance with the invention include, but are not limited to those listed in Table 4.

TABLE 4
Non-Immunoglobulin Factors Examples

| | |
|---|---|
| Growth Factors | EGF, FGF, IGF-1 IGF-2, TGF-α, TGF-β, PDGF, |
| | VEGF, NGF, CTGF, Growth Hormone, Insulin |
| Immunomodulators | Lactoferrin, |
| | Transferrin, Protease, PRP, IL-6, IL-8, |
| | IL-10, IF-γ, Lymphokines, Lysozyme, |
| | C3, C4, TNF specific to the host animal |
| Vitamins and | Vitamins B1, B2, B6, B12, E, A, C, Folic |
| Other Nutrients | Acid, Panthothenic Acid, Beta-carotene, |
| | Glycogen, Retinoic Acid |
| Minerals | Calcium, Chromium, Iron, Magnesium, |
| | Phosphorous, Potassium, Sodium, Zinc |
| Essential | Isoleucine, Leucine, Histidine, Methionine, |
| Amino Acids | Lysine, Threonine, Phenylalanine, Valine, |
| | Tryptophan |
| Nonessential | Arginine, Cysteine, Glutamic Acid, Alanine, |
| Amino Acids | Tyrosine, Glycine, Proline, Aspartic Acid, |
| | Serine |
| Additional | β-2 microglobulin, Haemopexin, Haptoglobulin, |
| Factors | Lactoperoxidase, Orotic Acid, Peroxidase, |
| | Xanthine Oxidase, Glycoproteins |

Key:
(−) = Negative regulation,
TGF = Transforming Growth Factor,
MCP = Macrophage Chemoattractant Protein,
MIP = Macrophage Inflammatory Protein,
GRO = Growth-Related Oncogene,
IL = Interleukin,
VEGF = Vascular Endothelial Growth Factor,
PLGF = Placenta Growth Factor,
FGF = Fibroblast Growth Factor,
HGF = Hepatocyte Growth Factor,
Cyr61 = Cysteine-Rich 61,
GM-CSF = Granulocyte-Macrophage Colony Stimulating Factor,
IP = Interferon-γ-Inducible Protein,
PDGF = Platelet-Derived Growth Factor,
CTGF = Connective Tissue Growth Factor,
IGF = Insulin-like Growth Factor,
NGF = Nerve Growth Factor,
EGF = Epidermal growth Factor,
HB-EGF = Heparin-Binding Epidermal Growth Factor,
NDF = Neu Differentiation Factors,
BMP = Bone Morphogenetic Proteins,
Ig = Immunoglobulin,
PRP = Proline-Rich Polypeptide,
C = Complement,
IF = Interferon-γ.

A polyclonal antibody composition of the invention that has been depleted of non-immunoglobulin factors are sometimes referred to herein as a “non-Ig factor-depleted polyclonal antibody compositions”. Such non-Ig factor-depleted polyclonal antibody compositions of the invention are suitable for use in the treatment of disease wherein the pathogenesis of the disease is modulated by a target antigen to which the polyclonal antibodies are directed. Such treatment also includes the mitigation of potential side effects associated with the use of polyclonal antibody compositions derived from a biological source in the treatment of disease whether the treatment is for acute disease or chronic disease.

The non-Ig factor-depleted polyclonal antibody compositions of the invention may be further processed to enrich for the presence of polyclonal antibodies specific for the target antigen wherein non-specific immunoglobulins have been selectively depleted or removed from the polyclonal antibody composition. Numerous techniques are known to those in the art for enriching polyclonal antibodies for antibodies to specific targets antigens. In one embodiment at least 60%, preferably at least 70%, preferably at least 80%, preferably at least 90%, and preferably at least 95% of the immunoglobulins present in a composition of the invention are polyclonal antibodies specific for a target antigen. In one embodiment, polyclonal antibody compositions are enriched for the target antigen such that the composition is substantially free of non-specific immunoglobulins. Non-Ig factor-depleted polyclonal antibody compositions that have been enriched for a target antigen are sometimes referred to herein as “enriched non-Ig factor-depleted polyclonal antibody compositions.” In one embodiment, the present invention comprises polyclonal compositions wherein non-specific antigens are depleted and non-immunoglobulin factors are optionally depleted.

In a preferred embodiment, the invention provides a composition comprising isolated and purified immunoglobulin derived from the colostrum of a bovine that has been immunized with all or a portion of a target antigen wherein the composition comprises polyclonal antibodies capable of binding the target antigen and/or neutralizing the target antigen and/or modifying the function of the target antigen in standard assays as are known in the art. Such assays include but are not limited to ELISA, radioimmunoassay, immunodiffusion, flow cytometry, Western blotting, agglutination, immunoelectrophoresis, surface plasmon resonance, and assays based on neutralization or modulation of the function of the target antigen, such as neutralization of TNF in the L929 cell-based assay. In one embodiment, the composition is at least 90% immunoglobulin as measured by reducing SDS PAGE/densitometry. In a preferred embodiment, the composition is at least 95%, preferably at least 97%, preferably at least 98% and preferably at least 99% immunoglobulin as measured by reducing SDS-PAGE/densitometry.

In one embodiment the isolated and purified immunoglobulin composition derived from bovine colostrum in accordance with the invention is depleted of non-immunoglobulin factors at least 5 fold below their normal levels in colostrum. In one embodiment the Ig composition is depleted of non-immunoglobulin factors at about 10 fold below their normal levels in colostrum. In one embodiment at least one of lactoferrin (LF), alpha-lactalbumin (a-Lac), beta-lactoglobulin (b-Lac), lactoperoxidase (LPO) and insulin-like growth factor-1 (IGF-1) is depleted at least 10 fold below its normal level in colostrum.

In one preferred embodiment, lactoferrin is present in the immunoglobulin composition derived from the colostrum of a bovine at a level of no more than about 10 mg per gram of total protein present in the composition wherein the total protein content of the composition is measured by bicinchonic acid (BCA) assay (Smith, P. K., et al., Measurement of protein using bicinchoninic acid. Anal. Biochem. 150, 76-85, (1985) and the level of lactoferrin is measured by ELISA. More preferably the level of lactoferrin is about 3 mg/g of total protein or less, more preferably about 1 mg/g of total protein or less and most preferably less than 1 mg/g total protein, such as 0.3 mg/g or less.

In one preferred embodiment, alpha-lactalbumin (a-Lac) is present in the Ig composition derived from the colostrum of a bovine at no more than about 75 mg/gram of total protein and preferably no more than about 20 mg per gram of total protein present in the composition wherein the total protein content of the composition is measured by bicinchonic acid (BCA) assay and the level of a-Lac is measured by ELISA. More preferably the level of a-Lac is about 3 mg/g (w/w) of total protein or less, more preferably about 1 mg/g or less of total protein and most preferably less than 1 mg/g total protein.

In one preferred embodiment, beta-lactoglobulin (b-Lac) is present in the Ig composition at no more than about 20 mg/g and preferably no more than about 10 mg per gram of total protein present in the composition wherein the total protein content of the composition is measured by bicinchonic acid (BCA) assay and the level of b-Lac is measured by ELISA. More preferably the level of b-Lac is about 5 mg/g or less of total protein, and more preferably about 3 mg or less of total protein, more preferably about 1 mg/g total protein or less and most preferably less than 1 mg/g total protein.

In one embodiment, lactoperoxidase (LPO) is present in the Ig composition at no more than about 10 mg per gram of total protein present in the composition wherein the total protein content of the composition is measured by bicinchonic acid (BCA) assay and the level of LPO is measured by ELISA. More preferably the level of LPO is about 2 mg/g (w/w) of total protein or less, more preferably about 1 mg/g total protein, more preferably about 0.2 mg/g total protein or less and most preferably less than 0.2 mg/g total protein.

In one embodiment, insulin-like growth factor-1 (IFG-1) is present in the Ig composition derived from the colostrum of a bovine at no more than about 10 mg per gram of total protein present in the composition wherein the total protein content of the composition is measured by bicinchonic acid (BCA) assay and the level of IGF-1 is measured by ELISA. More preferably the level of IFG-1 is about 1 mg/g of total protein or less, more preferably about 0.1 mg/g total protein or less and most preferably less than 0.1 mg/g total protein.

In one embodiment, the invention provides processes for preparing a composition comprising isolated and purified immunoglobulin derived from the colostrum of a bovine that has been immunized with all or a portion of a target antigen, wherein the composition is at least 90% immunoglobulin as determined by reducing SDS-PAGE/densitometry and is substantially depleted of non-immunoglobulin factors including but not limited to lactoferrin (LF), alpha-lactalbumin (a-Lac), beta-lactoglobulin (b-Lac), lactoperoxidase (LPO) and insulin-like growth factor-1 (IGF-1) wherein the composition binds a target antigen in standard antibody binding assays, wherein the preparation of the composition comprises the steps of: providing whey derived from the colostrum of a bovine immunized with a target antigen that has been processed to deplete the fat and casein by standard procedures as is known in the art; adjusting the pH of the processed whey to a pH of 6.6 to 7.0; filtering the whey through an anion exchange column connected in series with a cation exchange column wherein the whey sequentially flows through both columns connected in series without addition of materials that change the salt concentration or pH; collecting the flow through after it sequentially passes through both columns connected in series without addition of materials that change the salt concentration or pH before collection occurs; and concentrating the flow through by ultrafiltration. The process may further comprise lyophilizing or spray-drying the concentrated flow through product of step using standard techniques. The process may further comprise testing the concentrated flow through product to determine that the impurities are at desired levels prior to spray drying or lyophilizing by standard means including the assays described in the Examples.

In one embodiment, the anion exchange column is a strong anion exchanger and the cation exchange column is a strong cationic exchanger column. Strong cation exchangers suitable for use in this invention include but are not limited to Capto S (GE Healthcare Bio-Sciences, Piscataway, N.J.), ToyoPearl GigaCap S-650 M (Tosoh Bioscience, Tokyo, Japan), S Sepharose XL (GE Healthcare Bio-Sciences, Piscataway, N.J.), MacroPrep High S (Bio-Rad Laboratories, Hercules, Calif.), TSK Gel BioAssist S (Tosoh Bioscience, Tokyo, Japan), POROS XS (Life Technologies/Applied Biosystems, Carlsbad, Calif.). Strong anion exchangers suitable for use in this invention include but are not limited to Capto-Q (GE Healthcare Bio-Sciences, Piscataway, N.J.), ToyoPearl GigaCap Q-650 M (Tosoh Bioscience, Tokyo, Japan), Q Sepharose XL (GE Healthcare Bio-Sciences, Piscataway, N.J.), Macro-Prep High Q (Bio-Rad Laboratories, Hercules, Calif.), TSK gel BioAssist Q (Bio-Rad Laboratories, Hercules, Calif.), TSK gel QAE-25SW (Bio-Rad Laboratories, Hercules, Calif.), POROS HQ (Life Technologies/Applied Biosystems, Carlsbad, Calif.).

Weak cation and anion exchangers would also be suitable for use in this invention. Weak cation exchangers suitable for use in this invention include but are not limited to Macro-Prep CM (Bio-Rad Laboratories, Hercules, Calif.), CM Ceramic Hyper D (Pall Corporation, Port Washington, N.Y.), CM Sepharose FF (GE Healthcare Bio-Sciences, Piscataway, N.J.). Weak anion exchangers suitable for use in this invention include but are not limited to TSK-gel DEAE 5PW (Tosoh Bioscience, Tokyo, Japan), TSK-gel DEAE 5NPR (Tosoh BioScience, Tokyo, Japan), Capto-DEAE (GE Healthcare Bio-Sciences, Piscataway, N.J.), DEAE Ceramic Hyper-D (Pall Corporation, Port Washington, N.Y.), Mustang S (Pall Corporation, Port Washington, N.Y.), POROS D (Life Technologies/Applied Biosystems, Carlsbad, Calif.).

In one embodiment the conductivity of the whey solution entering the column is about 4+/−1 milliSiemens/cm. In one embodiment, the conductivity of the flow through of both columns is about 4+/−1 milliSiemens/cm. In one embodiment, the pH of the whey solution entering the column is the same as the pH of the flow through of both columns.

This method is particularly useful in the preparation of large scale amounts of a purified and isolated Ig composition of the invention substantially depleted of non-Ig factors as described above. Depletion of non-immunoglobulin factors from an Ig composition comprising, polyclonal antibodies using ion exchange chromatography has been challenging in the past due to the range of pIs of the various antibody clones within the polyclonal composition. Previous methods have required using multiple columns with varying conditions and elution steps to separate the immunoglobulin from the non-immunoglobulin factors having pIs above or below those of the polyclonal antibody species. The use of sequential flow through anionic and cationic ion exchange columns connected in series provide for large scale purification of polyclonal antibodies while simultaneously substantially depleting non-Ig factors from the final composition. This method allows for purification and isolation of Ig compositions without the need for of multiple columns, separate elutions and multiple changes in process conditions such as pH, salt and temperature. As used herein large scale purification means at least 30 L liters of starting material (colostrum).

In one preferred embodiment, the invention provides pharmaceutical formulations comprising an optional, pharmaceutically acceptable excipient as is described in detail herein and a composition consisting essentially of isolated and purified immunoglobulin derived from the colostrum of a bovine that has been immunized with all or a portion of a target antigen, wherein composition is at least 90% immunoglobulin as determined by reducing SDS-Page/densitometry and contains less than about 10 mg of lactoferrin per gram of total protein present in the composition wherein the total protein content of the composition is measured by bicinchonic acid (BCA) assay and the level of lactoferrin is measured by ELISA, wherein the composition binds or modulates the target antigen in an assay. The pharmaceutical compositions of the invention may be depleted of additional non-immunoglobulin factors as described above including but not limited to depletion of alpha-lactalbumin (a-Lac), beta lactoglobulin (b-Lac), lactoperoxidase (LPO) and insulin-like growth factor-1 (IGF-1) to the levels as described herein.

The purified and isolated immunoglobulin compositions derived from the colostrum of a bovine in accordance with the invention may comprise polyclonal antibodies specific for any target antigen for example, antigens associated with disease pathology or the treatment of disease. For example, the non-Ig factor-depleted polyclonal antibody compositions of the invention may be directed at biological targets expressed on or near the luminal surface of the digestive tract as well as below the mucosal barrier such as on the basal side of the epithelium, targets expressed in the submucosa, target expressed in the lateral intercellular space, and targets expressed in the lamina propria. For the purposes of the invention, the “digestive tract” consists of the mouth, pharynx, esophagus, stomach, small intestine (duodenum, jejunum, ileum), large intestine (cecum, colon, rectum) and anus.

In one embodiment, polyclonal antibodies present in the compositions of the invention cross the mucosal barrier of the patient as a result of pre-existing damage to the mucosal barrier. In one embodiment, the mucosal barrier of the digestive tract may be breached or compromised through mechanical trauma, including but not limited to dental and oral wounds, esophageal wounds, or surgically induced trauma due to partial gut resection, jejunostomy, ileostomy, colostomy or other surgical procedures. The mucosal barrier of the digestive tract may also be breached by ischemia or reperfusion injury. The mucosal barrier of the digestive tract may also be breached by damage caused by cancer chemotherapy, cancer radiation therapy, or high dose radiation exposure outside of a therapeutic setting. The mucosal barrier of the digestive tract may be breached or compromised through gross inflammation and/or ulceration, including but not limited to periodontal disease, aphthous stomatitis, bacterial, viral, fungal or parasitic infections of the digestive tract, peptic ulcers, ulcers associated with stress or H. pylori infection, damage caused by esophageal reflux, inflammatory bowel disease, damage caused by cancer of the digestive tract, food intolerance, including celiac disease, or ulcers induced by non-steroidal anti-inflammatory drugs (NSAIDs) or other ingested or systemically delivered drugs.

In one embodiment of the invention, polyclonal antibodies are specific for target antigens such as cytokines that regulate inflammation, including but not limited to TNF, TNF-kappa, Ifn-gamma, IL-1 beta, IL-2, IL-6, IL-12, IL-13, IL-15, IL-17, IL-18, IL-21, IL-23, IL27, IL-32, IL-33 and IL-35. In one embodiment of the invention, polyclonal antibodies are specific for target antigens that are enteric neurotransmitters or their receptors or transporters expressed below the mucosal barrier of the digestive tract, including receptors for serotonin that are expressed in the gut (5-HT1A, 5-HT1B/B, 5-HT2A, 5-HT2B, 5-HT3, 5-HT4, 5-HT7, 5-HT1P). In one embodiment of the invention, polyclonal antibodies of the invention are specific for target antigens that are peptides that regulate food intake or the receptors for such peptides. Such peptides include but are not limited to CCK, GLP1, GIP, oxyntomodulin, PYY3-36, enterostatin, APOAIV, PP, amylin, GRP and NMB, gastric leptin and ghrelin. In one embodiment of the invention, polyclonal antibodies of the invention are specific for target antigens that are epidermal growth factor receptors on colorectal cancer cells. In one embodiment, polyclonal antibodies of the invention are specific for target antigens that are biological targets that enhance wound healing, that alter the function of tight junctions such as occludin, claudins, junctional adhesion molecule, ZO-1, E-cadherin, coxackie adenovirus receptor and serine proteases such as elastase that are involved in the release of claudins.

In one embodiment, polyclonal antibodies of the invention are specific for target antigens that are apical intestinal receptors. “Apical intestinal receptors” as used herein are endogenous transmembrane proteins, expressed in the cell membrane of cells facing the luminal side of the intestinal tract. Classes of apical intestinal receptors described in this invention include but are not limited to: nutrient receptors and transporters (including sugar receptors and transporters, taste receptors, amino acid transporters, and free fatty acid receptors); pattern recognition receptors (including the Toll-like receptors); chemokine and cytokine receptors; bile salt transporters; transporters for calcium iron, and other ions and minerals; peptidases; disaccharidases; growth factor receptors (including epidermal growth factor receptor) and proteins expressed on the surface of cancerous cells in the GI tract. Apical intestinal receptors may be expressed in the stomach, the small intestine or the colon.

In one embodiment, polyclonal antibodies of the invention are specific for target antigens that are food antigens. Such polyclonal antibodies are useful in the treatment or prevention of food allergies or intolerances, including celiac disease. In one embodiment, polyclonal antibodies of the invention are specific for target antigens that are gluten or gluten derived peptides and are useful for treatment of celiac disease.

In one preferred embodiment, non-Ig factor-depleted polyclonal antibody preparations of the invention comprise polyclonal antibodies that are specific for the inflammatory cytokine, TNF-alpha “TNF”. Such compositions are sometimes referred to herein as “non-Ig factor-depleted anti-TNF polyclonal antibody compositions”. Patients with Crohn's disease and ulcerative colitis collectively referred to in the art as inflammatory bowel disease are frequently treated with systemically administered antibodies (e.g. monoclonal antibodies) directed against the TNF. In one preferred embodiment, the invention comprises pharmaceutical compositions and methods for treating inflammation, and particularly inflammatory bowel disease using non-Ig factor-depleted anti-TNF polyclonal antibody compositions of the invention, and preferably bovine milk-derived or bovine colostrum-derived pharmaceutical compositions of the invention. Such non-Ig factor-depleted anti-TNF polyclonal antibody compositions of the invention may be further depleted of non-specific antibodies in accordance with the invention.

In one embodiment, non-Ig factor-depleted anti-TNF polyclonal antibody compositions of the invention are suitable for use in the treatment of oral or intestinal mucositis. The mucositis may, for example, be caused by radiation therapy, chemotherapy or any combination thereof. In one embodiment, the mucositis may be caused by exposure to high doses of radiation, including total body irradiation, outside of the context of radiation therapy. In one embodiment, non-Ig factor-depleted anti-TNF polyclonal antibody compositions of the invention are suitable for use in the treatment of recurrent aphthous stomatitis. Compositions of the invention, may be administered topically, to the oral cavity to treat oral mucositis and aphthous stomatitis, or orally or rectally to the digestive tract to treat intestinal mucositis. Such formulations are well known to those skilled in the art. These routes of administration and dosage forms are discussed in detail herein.

In one aspect, the invention provides methods of treating a patient using the polyclonal antibody compositions and formulations of the invention. The term “patient” as used herein refers to an animal. Preferably the animal is a mammal. More preferably the mammal is a human. A “patient” also refers to, for example, dogs, cats, horses, cows, pigs, guinea pigs, fish, birds, reptiles and the like.

The terms “treatment” “treat” and “treating” encompasses alleviation, cure or prevention of at least one symptom or other aspect of a disorder, disease, illness or other condition (collectively referred to herein as a “condition”), or reduction of severity of the condition, and the like. A composition or pharmaceutical formulation of the invention need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. In one embodiment, an indication that a therapeutically effective amount of a composition has been administered to the patient is a sustained improvement over baseline of an indicator that reflects the severity of the particular disorder.

The pharmaceutical formulations of the invention are preferably administered to the patient by topical administration to the oral cavity including sublingual and submucosal administration; intranasal administration; oral administration to the digestive tract, rectal administration or by inhalation.

Most preferably, for disorders of the oral cavity, the antibodies of the invention can be delivered in a mouthwash, rinse, paste, gel, or other suitable formulation. Compositions of the invention can be delivered using formulations designed to increase the contact between the active antibody and the mucosal surface, such as buccal patches, buccal tape, mucoadhesive films, sublingual tablets, lozenges, wafers, chewable tablets, quick or fast dissolving tablets, effervescent tablets, or a buccal or sublingual solid.

Most preferably, for disorders wherein delivery to the digestive tract is most effective, compositions and formulations of the invention can be delivered by oral ingestion in the form of a capsule, tablet, liquid formulation or similar form designed to introduce drug to the digestive tract. Alternatively, formulations and compositions of the invention may be administered by suppository or enema for delivery to the lower digestive tract. Such formulations are well known to those skilled in the art. These routes of administration and dosage forms are discussed in detail herein.

The pharmaceutical formulations of the present invention are optionally formulated together with one or more pharmaceutically acceptable carriers or excipients. By a “therapeutically effective amount” of a polyclonal antibody composition of the invention is meant an amount of the composition which confers a therapeutic effect on the treated subject, at a reasonable benefit/risk ratio applicable to any medical treatment. The therapeutic effect is sufficient to “treat” the patient as that term is used herein.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Pharmaceutically acceptable excipients include those that are used to prevent protein aggregation and/or provide thermostability including such as polyols, sugars and proteins, including, but not limited to: sorbitol, mannitol, glycerol, trehalose, maltose, glutamic acid, arginine, and histidine.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. In one embodiment, compositions for rectal administration are in the form of an enema.

Solid dosage forms for oral administration include capsules, tablets, pills, powders, sachets and granules. In such solid dosage forms, the active compound may be mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

It may be desirable under some conditions to provide additional levels of protection against gastric degradation. If this is desired, there are many options for enteric coating (see for example U.S. Pat. Nos. 4,330,338 and 4,518,433). In one embodiment, enteric coatings take advantage of the post-gastric change in pH to dissolve a film coating and release the active ingredient. Coatings and formulations have been developed to deliver protein therapeutics to the small intestine and these approaches could be adapted for the delivery of an antibody of the invention.

In addition, the solid dosage forms of tablets, dragees, capsules, pills, and granules can be prepared with other coatings and shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Effective doses will vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific composition employed; the specific composition employed; the age, body weight, general health, sex and diet of the patient; the time of administration, route of administration, and rate of excretion of the specific composition employed; the timing of delivery of the compound relative to food intake; the duration of the treatment; drugs used in combination or contemporaneously with the specific composition employed; and like factors well known in the medical arts.

In accordance with the invention, routes of administration include oral administration via catheter or feeding tube.

Particular embodiments of the present invention involve administering a polyclonal composition of the invention such that the dosage of polyclonal antibody is from about 1 mg per day to about 1 g/day, more preferably from about 10 mg/day to about 500 mg/day, and most preferably from about 20 mg/day to about 100 mg/day, to a subject. In one embodiment, a polyclonal antibody composition is administered such that the dosage of polyclonal antibody is from about 100 mg to about 50 g/day, more preferably from about 500 mg/day to about 10 g/day, and most preferably from about 1 g/day to about 5 g/day, to a subject. In one embodiment lower dosages may be used when the composition has been enriched for polyclonal antibodies directed to the target antigen.

Treatment regimens include administering an antibody composition of the invention one time per day, two times per day, or three or more times per day, to treat a medical disorder disclosed herein. In one embodiment, an antibody composition of the invention is administered four times per day, 6 times per day or 8 times per day to treat a medical disorder disclosed herein. In one embodiment, an antibody composition of the invention is administered one time per week, two times per week, or three or more times per week, to treat a medical disorder disclosed herein.

The methods and compositions of the invention include the use of non-Ig factor-depleted polyclonal antibody compositions of the invention in combination with one or more additional therapeutic agents useful in treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an antibody of the invention is administered at least once during a course of treatment that involves administering at least one other therapeutic agent to the patient.

The following examples are provided for the purpose of illustrating specific embodiments or features of the invention and are not intended to limit its scope.

### EXAMPLES

### Example 1

### Bovine Colostral Anti-TNF Polyclonal Antibody Composition and Process

Immune colostrum is produced at an audited, qualified animal facility. Pregnant Holstein dairy cows are sourced from commercial Grade A dairies in the US which are regulated under the FDA Pasteurized Milk Ordinance (PMO). The PMO specifies housing requirements, building and equipment standards, use of acceptable cleaning and pesticide materials, milking procedures, sanitation requirements, etc.

Animals are quarantined for a minimum of two weeks prior to start of immunizations and dried off if necessary. Qualified cows are housed and maintained separately from other animals and observed daily. Feed source are controlled to prevent the introduction of unapproved animal source protein. Source dairy herds are tested or certified by the state to be free of brucellosis and TB. Cows receive (killed or inactivated) routine immunizations for, or are screened for:

| | |
|---|---|
| Bovine leukemia virus | E. coli |
| Bovine viral diarrhea virus | Rotavirus |
| Parainfluenza virus (PI₃) | Vibriosis |
| Infectious bovine rhinotracheitis | Leptospirosis |
| Bovine respiratory syncytial virus | Clostridial diseases |
| Mycobacterium paratuberculosis | Coxiella burnetti. |

Qualified cows are immunized with three (3) doses of rhTNF using commercial veterinary adjuvants that have been USDA approved for use in dairy cows. Final prepared vaccines are administered under the direct supervision of a veterinarian according to established SOPs at intervals of two-three weeks. Serum samples are collected at the time of each injection and at calving.

Immunized cows are milked individually. Animals must be in apparent good health at calving with no evidence of clinical mastitis. The cow's udder is prepared for milking using standard dairy cleaning practices and materials approved for use under the FDA Pasteurized Milk Ordinance. Colostrum is collected twice daily for three (3) days after parturition. A sample of each individual colostral milking is collected for analysis and both samples and bulk colostrum are immediately frozen at −20° C. All incoming raw colostrum is qualified before use.

Colostrum is thawed and the fat component is reduced by continuous flow centrifugation at a flow rate of 1200 to 3600 lb/hr and a temperature of 24° to 43.5° C. The skim is diluted with 1.5 volumes of reverse osmosis (RO) water, the pH measured and recorded, and then adjusted to 4.6±0.1 with acid. The acidified skimmed colostrum is allowed to remain quiescent for 25-45 minutes at a temperature of 21° to 35° C. The casein precipitated by the acidification step is removed by decanting centrifugation. Clarified supernatant and casein sludge are collected separately, measured and recorded, and the casein fraction discarded.

Immunoglobulins from the clarified supernatant are isolated by Protein G chromatography in a closed system. Protein G resin (e.g. Sepharose 4 Fast Flow gel, Pharmacia Biotech AB, Uppsala, Sweden), is packed into a column and equilibrated with binding buffer as recommended by the manufacturer. To ensure proper ionic strength and pH are maintained for optimal binding, the clarified supernatant is dialyzed against binding buffer and then applied to the bed volume at a ratio of total protein to bed volume of 20 mg/ml. Flow rate is 0.8 ml/min. The column is washed with 10 bed volumes of the binding buffer. Bound bovine IgG is eluted with 10 bed volumes of 0.1 M glycine-HCl buffer (pH 2.7). To neutralize the eluted fractions, 100 μl/ml of 1M Tris-HCl (pH 9.0) is added to the collection tubes prior to the elution. The purification profile is monitored at 280 nm and target fractions collected, pooled and dialyzed against PBS at 4° C. The collected product eluate is concentrated by ultrafiltration.

### Example 2

### Bovine Colostral Anti-TNF Antibody: Comparison of Purified Antibody with Immune Colostral Whey in a Mouse Model of Inflammatory Bowel Disease

Immune colostrum was produced at Southwest Biolabs, a USDA-registered research facility in Las Cruces, N. Mex. Six Holstein cows were purchased during their last trimester of pregnancy, transported to the facility, and acclimatized for one week prior to immunization. The animals received 3 subcutaneous injections of antigen with one of two adjuvants, spaced 2-3 weeks apart, with the last injection given three weeks prior to the calculated date of parturition. Colostrum was collected from all animals for the first 8 milkings (first four days after calving). One animal calved prematurely, before full udder development had occurred, resulting in low levels of immunoglobulin in the colostrum, and colostrum from this animal was discarded.

A pool was prepared from colostrum collected on days 1-4 post-parturition and whey was prepared using standard methods (Su and Chiang, 2003). Colostrum was diluted 1:3 with distilled water, acidified to pH 4.6 with glacial acetic acid to precipitate casein, and centrifuged. The supernatant was removed and the pH was adjusted to 7.4 to generate immune whey.

The immunoglobulin fraction was purified using thiophilic adsorbent. Thiophilic adsorbent (T-gel) was purchased from Pierce (Thermo Scientific). A chromatography column was packed with 50 ml of resin and equilibrated with 150 ml binding buffer (0.5 M sodium sulfate, 20 mM sodium phosphate, pH 8.0). Immune whey was thawed in a water bath and solid sodium sulfate added to bring the final concentration to 0.5 M. The solution was spun at 3700 rpm for 15 minutes to remove particulate matter, diluted 1:1 with binding buffer and loaded onto the T-gel column at room temperature. The column was washed with 5 column volumes (150 ml) of binding buffer. Immunoglobulin was eluted with low salt (50 mM sodium phosphate pH 8.0) and column fractions containing protein were eluted and pooled. The eluted material was concentrated on an Amicon stirred cell with a YM filter with a 100,000 molecular weight cutoff and filter sterilized.

Control immunoglobulin was purified in parallel. Both immunoglobulin containing anti-TNF activity (immune immunoglobulin, AVX-470m) and control colostral immunoglobulin were assayed for their ability to both bind to and neutralize murine TNF. Immune immunoglobulin bound to TNF in a specific ELISA, while no binding was seen with control immunoglobulin.

The ability of the bovine antibody to neutralize TNF was determined using a standard cell-based TNF assay using murine L929 cells. Varying concentrations of antibody were preincubated with murine TNF for 2 hr at 37° C. in a 96 well microtiter plate. The antibody-antigen mixture was added to confluent cultures of L929 cells along with 1 ug/ml actinomycin D and incubated at 37° C. for 24 hr. Cell viability was assessed using the WST assay. Anti-TNF antibody neutralized TNF in this cell based assay, while the control antibody had no effect.

The purified AVX-470m and control immunoglobulin, along with whey from cows immunized with murine TNF and control whey, were evaluated in the murine TNBS-induced colitis model. The study was performed at Biomodels, LLC. Male C57Bl/6 mice with average starting body weight of 21.0 g were obtained from Charles River Laboratories (Wilmington, Mass.). Mice were acclimatized for 5 days prior to study commencement. Colitis was induced by the intrarectal administration of 4 mg of TNBS in a 50% ethanol vehicle on day 0.

Colitis was induced by intrarectal administration of 100 μL of TNBS (4 mg) in 50% ethanol under isoflurane anesthesia on day 0. Eight additional animals served as untreated controls and were dosed intrarectally with 100 μL of 50% ethanol. Animals were dosed with test article or vehicle twice a day (b.i.d.) at 0.1 mL per dose, from day −1 to day 3 via oral gavage (p.o.). On day 5 colitis severity was assessed in all animals using video endoscopy. Endoscopy was performed in a blinded fashion using a small animal endoscope (Karl Storz Endoskope, Germany). To evaluate colitis severity, animals were anesthetized with isoflurane and subjected to video endoscopy of the lower colon. Colitis was scored visually on a scale that ranges from 0 for normal, to 4 for severe ulceration. In descriptive terms, this scale is defined as follows:

### Endoscopy Colitis Scoring Scale

### Score: Description

### 0: Normal

1 Loss of vascularity
2: Loss of vascularity and friability
3: Friability and erosions
4: Ulcerations and bleeding
Statistical differences between a test group and the vehicle control were determined using a Student's t-test (SigmaPlot 11.2, Systat Software, Inc.).
The endoscopy scores are shown below.

| | | | Difference from |
|---|---|---|---|
| | | | TNBS-vehicle |
| | Ave | St Dev | control |
| EtOH control | 0.25 | 0.46 | p < 0.001 |
| TNBS - vehicle | 2.17 | 0.58 | NA |
| 5 mg AVX-470m | 1.50 | 0.76 | p = 0.038 |
| 1.5 mg AVX-470m | 1.75 | 0.71 | NS |
| 0.5 mg AVX-470m | 1.75 | 1.28 | NS |
| 1.5 mg Control Ig | 2.50 | 0.76 | NS |
| AVX-470 m whey | 1.63 | 0.52 | p = 0.046 |
| Control whey | 2.00 | 0.53 | NS |

Colitis scores were significantly elevated in the groups treated with TNBS compared to the ethanol-treated control group. Groups receiving oral treatment with 5 mg AVX-470m or AVX-470m whey both displayed significantly reduced colitis severity scores on day 5. No other significant differences in colitis severity were observed.

Surprisingly, these data demonstrate that activity is seen both with AVX-470m and with purified AVX-470m; no diminution of activity is seen when the immunoglobulin is purified away from the other whey components.

### Example 3

### Production of Immune Colostrum

Immune colostrum was produced at an audited, qualified animal facility. Pregnant Holstein dairy cows were sourced from commercial dairy farms regulated under the US FDA Grade A Pasteurized Milk Ordinance (PMO).

Animals were quarantined and dried off Source dairy herds were tested or certified by the state to be free of brucellosis and TB. Cows received (killed or inactivated) routine immunizations for, or were screened for:

| | |
|---|---|
| Bovine leukemia virus | E. coli |
| Bovine viral diarrhea virus | Rotavirus |
| Parainfluenza virus (PI₃) | Vibriosis |
| Infectious bovine rhinotracheitis | Leptospirosis |
| Bovine respiratory syncytial virus | Clostridial diseases |
| Mycobacterium paratuberculosis | Coxiella burnetti. |
| Bovine rabies | |

Qualified cows were immunized with three (3) doses of rhTNF using Quil A adjuvant at two to three week intervals with the last injection given three weeks prior to the calculated date of parturition. Colostrum was collected from all animals for the first 8 milkings (first four days after calving). A sample of each individual colostral milking was collected for analysis and both samples and bulk colostrum were immediately frozen at −20° C. All cows produced specific antibody as judged by specific binding to recombinant human TNF by ELISA and neutralization of recombinant human TNF in the L929 cell assay.

### Example 4

### Purification of Immunoglobulin from Bovine Colostrum by Ammonium Sulfate Precipitation

Colostrum samples from cows immunized with recombinant murine TNF were thawed and combined to generate a pool of 750 mL of colostrum. To remove fat, the colostrum was centrifuged at 2954×g for 20 minutes at room temperature. After fat removal, the colostrum was diluted in water (1 part colostrum; 2 parts water), and the pH was adjusted to 4.6 using acetic acid, then stirred for 20 minutes. The suspension was centrifuged at 3488×g for 30 minutes at room temperature and the casein pellet was removed from the whey. The pH of the whey was adjusted to pH 7.4 using 10N NaOH. A 50% saturated ammonium sulfate solution (313 g/L of ammonium sulfate) was slowly added to the whey and stirred for 1.5 hours at 4° C. The suspension was centrifuged at 3488×g for 30 minutes at 4° C. The supernatant was slowly decanted. The immunoglobulin pellet was resuspended in phosphate buffered saline (PBS, pH 7.2) to dissolve the pellet. The samples were dialyzed against 8 changes of 2 L of PBS (pH 7.2) at 4° C. for 36 hours. Bovine immunoglobulin was concentrated by adding polyvinylpyrrolidone powder (PVP-40, SIGMA-Aldrich, St Louis, Mo.) on top of the tubes at 4° C. The concentrated immunoglobulin solution was removed from the dialysis tubes.

### Example 5

### Removal of an Impurity from Colostrum on a HiTrap Capto S Column

Frozen colostrum (1.89 L) was thawed in a water bath at 45° C. Following an acidification step with acetic acid to precipitate casein, the colostrum preparation was held overnight at 4° C. The acidified material was warmed to 43° C. and centrifuged at 2,730 RCF. The supernatant was retained and neutralized to pH 6.4 with sodium hydroxide. The neutralized preparation was diluted by adding an equal volume of reverse osmosis water to produce 2.8 L of defatted, casein-reduced colostrum or colostral whey. Aliquots of the whey preparation were tested to evaluate the effectiveness of various chromatography columns.

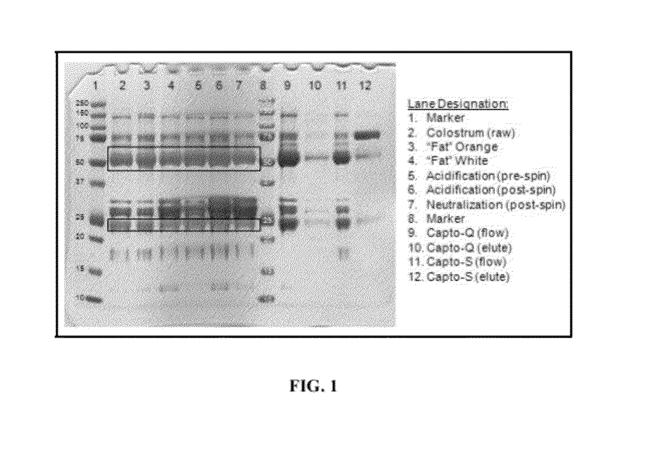

In this example, an aliquot (30 mL) of the whey was applied to an anion exchange column (5 mL HiTrap Capto Q packed column, purchased from GE Healthcare Bio-Sciences, Piscataway, N.J.) or a cation exchange column (5 mL HiTrap Capto S, also from GE Healthcare Bio-Sciences). Each column was eluted with 1 M NaCl, and the flow through and eluate were analyzed by SDS-PAGE under reducing conditions. Marker lanes were loaded with Dual Color Molecular Weight Marker (Bio-Rad Laboratories, Hercules, Calif.). The gel was stained with Coomassie Blue R-250 to visualize proteins. The gel is shown in FIG. 1. Lanes 1-7 represent the preparation of the whey component and lanes 9-12 illustrate the results of column chromatography. The two bands at 50 kDa and 25 kDa that are boxed indicate the heavy and light chains, respectively, of immunoglobulin. During the preparation of the whey, there is no significant yield loss of immunoglobulin as judged by this method (lanes 1-7). Immunoglobulin is visualized in the flow through of both the Capto-Q and Capto-S columns. Under the conditions used for this colostral whey preparation, the Capto-Q matrix did not significantly bind any abundant protein in the whey preparation, although it can have an important role in removal of less abundant protein impurities, as seen in further examples. By contrast, Capto-S was noted to bind and thus concentrate a protein from colostral whey with a reduced molecular weight of approximately 75 kDa (lane 12).

### Example 6

### Preparation of Polyclonal Antibody Composition by Depleted of Non-Immunoglobulin Factors by Mercapto-Ethyl-Pyridine (MEP) Chromatography

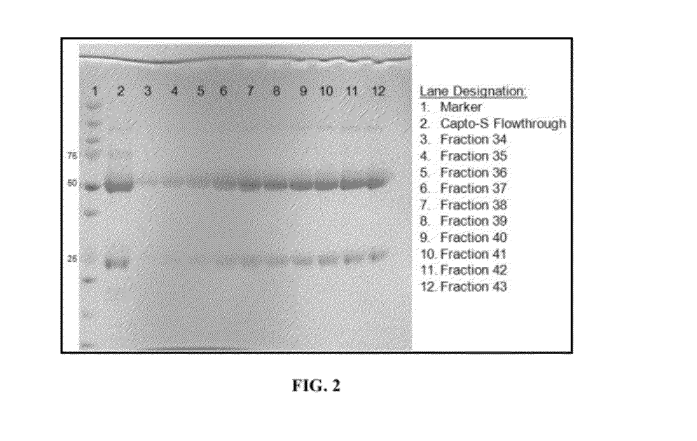

MEP matrix (Pall Corporation, Port Washington, N.Y.), useful for the purification of immunoglobulins, was tested for its ability to purify the polyclonal antibody preparation from whey. In this example, a 25 mg sample from the Capto-S flow through was adjusted to a final concentration of 0.15 M NaCl and filtered with an 0.22 μm filter (Millipax, Millipore Corporation, Billerica, Mass.). The sample was then applied to a 1 mL column of MEP matrix at a flow rate of 2 mL/min. Absorbance at 280 nm was monitored, and the column was washed until absorbance units reached baseline levels. Protein that bound to the column was eluted with a gradient of citric acid to decrease the pH. The immunoglobulin fraction eluted at approximately pH 5.0. In FIG. 2, Lane 1 is the BioRad Precision Dual Color marker, Lane 2, the Capto-S flow through (MEP column load), Lanes 3-12 fraction 36-43 inclusive fractions from the elution peak. A densitometry scan quantitated using ImageJ software (NIH, http://rsb.info.nih.gov/ij/index.html) revealed that the heavy and light chains accounted for approximately 95% of the total protein.

These data suggest that MEP may be an effective resin for removing impurities. However, later examples will demonstrate that MEP is not the preferred method.

### Example 7

### Investigation of the Composition of the MEP-Purified Whey Protein Antibody Preparation by Analytical Size Exclusion Chromatography

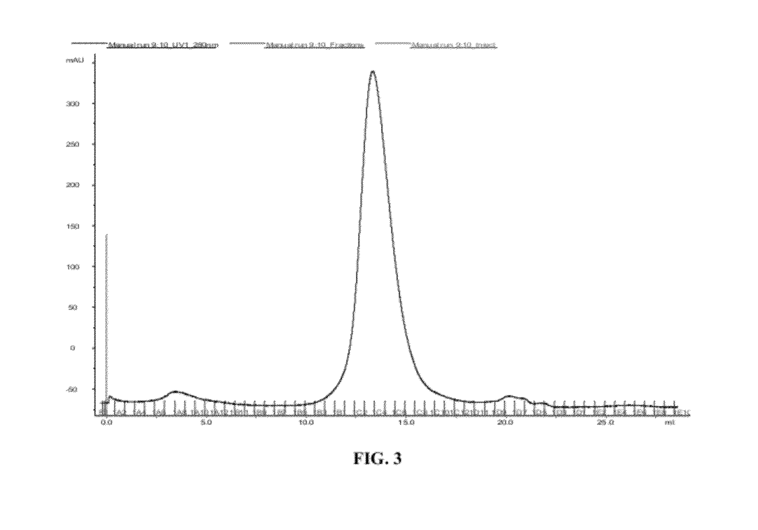

Size exclusion chromatography is a useful technique for assessing the composition of purified protein preparations. Protein complexes or proteins with higher native molecular weight elute earlier than proteins with lower native molecular weight. Pooled MEP eluate from the chromatography of whey protein (0.5 mg in a total volume of 0.5 mL) was subjected to analytical size exclusion chromatography analysis on a high resolution TRICORN®S200 Column (Superdex 200 10/300 GL, from GE Healthcare Bio Sciences, Piscataway, N.J.) on an ÄKTAEXPLORER™ FPLC system. The column was pre-equilibrated in phosphate buffered saline (0.15M NaCl), which was also the elution buffer. Absorbance was monitored at 280 nm. Area under the peaks was measured using the Unicorn software package. Under these conditions, the immunoglobulins were expected to maintain native conformation. As shown in FIG. 3, a primary peak with elution volume of 13.5 mL was calculated to represent a retention time of approximately 149 kDa for a globular protein, very close to the theoretical molecular weight 150 kDa molecular weight for an immunoglobulin. The data in this example are consistent with the SDS-PAGE analysis and show that the MEP matrix bound and purified the polyclonal antibody composition.

### Example 8

### Investigation of Scale Up to Larger Scale Process Using 15 L Colostrum and a 2 L MEP Column

In this example, parameters were investigated in order to scale up the MEP column process. Defatted whey was prepared at pilot scale: first, defatted colostrum (15 L) was prepared by continuous flow centrifugation, followed by acidification to pH 4.6 with 10% lactic acid. After an overnight hold, the casein was removed by centrifugation and the supernatant was retained and neutralized to pH 6.4 with 0.5 M NaOH. The whey was then filtered through a pilot scale filter train, a depth filter (CUNO Zeta Plus filter Cartridge) followed by a 0.2 μm filter, and loaded onto a 2 L column of MEP resin packed into an INdEX column preequilibrated with 20 mM citrate-phosphate buffer, pH 6.8. The column was extensively washed with approximately 10 L of the same buffer, and then eluted with 20 mM citrate-phosphate, pH 2.8. The eluted sample was neutralized with 1 M Tris. The eluate was then diafiltered versus 5 volumes of reverse osmosis water to exchange the buffer, and then concentrated by ultrafiltration using a Pilot Scale Tangential Flow Filtration Apparatus (Pall Corporation, Port Washington, N.Y.). Viscosity was not observed to be a problem.

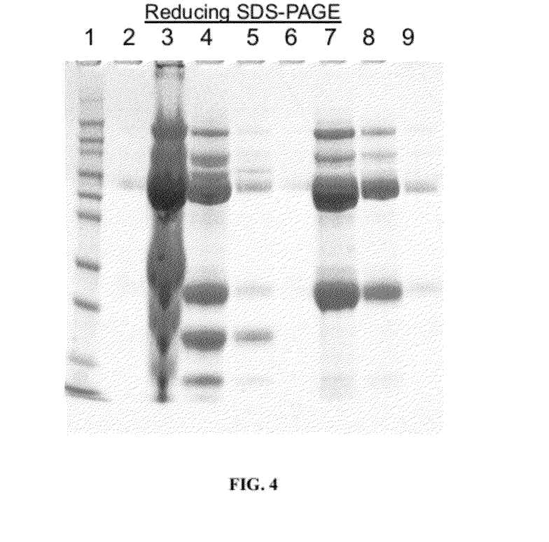

The reducing SDS PAGE analysis shown in FIG. 4 is a volume-loaded gel for rapid analysis (resulting in some lane overloading). Lane 1, markers, Lane 2, blank, Lane 3, MEP Load, Lane 4, MEP flow through, Lane 5, MEP Wash, Lane 6, blank, Lane 7-9 MEP eluate fractions. These results largely recapitulated the results seen at the bench scale. Two major impurities were noted to be present in addition to immunoglobulin heavy and light chain.

### Example 9

### Spray Drying Example from 12-19-2011 Report

Eluate from MEP chromatographic separation of bovine immunoglobulin was concentrated by ultrafiltration/diafiltration to approximately 80 mg/ml protein to create the feedstream for bench scale spray drying experiments. All spray drying development work was conducted by Pharma Spray Drying, Inc. Bedford Hills, N.Y., using a Buchi B-290 bench top lab spray dryer.

The purpose of these initial experiments was to identify spray drying conditions that would form a collectable powder within the cyclone with minimum sticking and product hold up. No excipients were added to the concentrated colostral immunoglobulins prior to spray drying.

Buchi B-290 Test Work

| | Inlet | Outlet | Atm. Air | | | | |
|---|---|---|---|---|---|---|---|
| Test | Temp. | Temp. | Pressure | Fan | Atm. Air | Pump | |
| # | Deg. C. | Deg. C. | (bar) | speed | Rate | setting | Results |
| 1 | 110 | 65 | 6 | 100% | 30% | 25 rpm | 1.46 g collected. |
| | | | | | | | Great collection no |
| | | | | | | | sticking in cyclone |
| 2 | 110 | 75 | 6 | 100% | 30% | 10 rpm | 1.25 g collected. |
| | | | | | | | Great collection no |
| | | | | | | | sticking in cyclone |
| 3 | 100 | 60 | 6 | 100% | 30% | 18 rpm | 3.1 g collected. |
| | | | | | | | Great collection |
| | | | | | | | slight sticking in |
| | | | | | | | cyclone |
| 4 | 100 | 50 | 6 | 100% | 30% | 22 rpm | 4.9 g collected. |
| | | | | | | | Great collection |
| | | | | | | | slight sticking in |
| | | | | | | | cyclone |
| 5 | 120 | 80 | 6 | 100% | 30% | 15 rpm | 2.5 g collected. |
| | | | | | | | Great collection no |
| | | | | | | | sticking in cyclone |
| 6 | 120 | 70 | 6 | 100% | 30% | 25 rpm | 1.9 g collected. |
| | | | | | | | Great collection no |
| | | | | | | | sticking in cyclone |
| 7 | 120 | 60 | 6 | 100% | 30% | 32 rpm | 3.4 g collected. |
| | | | | | | | Slight overspray |
| 8 | 120 | 50 | 6 | 100% | 30% | 47 rpm | 2.8 g collected. |
| | | | | | | | Over spraying in |
| | | | | | | | main |
| 9 | 150 | 90 | 6 | 100% | 30% | 18 rpm | 3.0 g collected. |
| | | | | | | | Great collection no |
| | | | | | | | sticking in cyclone |
| 10 | 150 | 75 | 6 | 100% | 30% | 42 rpm | 2.3 g collected. |
| | | | | | | | Great collection no |
| | | | | | | | sticking in cyclone |
| 11 | 100 | 55 | 6 | 100% | 30% | 28 rpm | 2.5 g collected. |
| | | | | | | | Great collection |
| | | | | | | | slight sticking in |
| | | | | | | | cyclone |

Each of these test powders was hand-filled into gelatin capsules (Size 00, Capsugel, Cambridge, Mass.) to produce prototype oral dosage forms.

### Example 10

### Comparison of MEP Purification and Capto-S Purification Processes

In evaluating the results obtained using the MEP resin, there was concern about the presence of impurities in the eluate, as well as concerns about binding capacity. In addition, in a process for preparation of pharmacologic compositions, scalability, rapid throughput, and avoiding changes in volume are important factors. A process whereby the active pharmaceutical ingredient does not bind to a column resin while undesired contaminants do bind may represent a preferred process. Therefore, the flow through methods were re-examined.

In this example, early steps are performed as described (Gregory, A. G., U.S. Pat. No. 5,707,678): defatted colostrum was diluted 2× with reverse osmosis water, acidified, neutralized, then processed in the continuous flow centrifuge. After an overnight hold step, diatomaceous earth (USP/NF grade, Sigma Aldrich) was added to 4/g L and the material was stirred for 10 min, neutralized with 10% sodium hydroxide, and filtered through a Cuno Zeta Plus BioCap depth filter (602A05A, 3M Corporation, St. Paul, Minn.) and a 0.2 μm filter (MilliPAK MPGL 02GH2, Millipore Corporation, Billerica, Mass.).

The whey was applied to either an MEP column or Capto-S column. Following chromatography, the appropriate fractions from each arm of the comparison (retained fractions, eluted with a pH gradient for MEP; flow through for Capto-S, adjusted to 100 mM NaCl) were then ultrafiltered to an estimated concentration of 50 g/L using a Pall Pharmaceutical series apparatus (Pall Corporation, Port Washington, N.Y.) and TMP-Flux 50 kD nominal molecular weight cut-off (NMWCO) membranes. The trans-membrane pressure (TMP) was adjusted to maintain a level close to 15 psi. The material was diafiltered versus three to five volumes of reverse-osmosis water, followed by a second ultrafiltration step to bring the protein concentration to 100 g/L. Protein concentration was determined by the bicinchoninc acid method using the BCA™ assay kit, carried out as described by the supplier (Thermo Fisher Scientific, Rockford, Ill.). Samples were run on reducing 4-12% Bis-Tris NOVEX Gels (NUPAGE, Invitrogen) using NUPAGE MOPS SDS Running Buffer. Marker lanes were Novex Sharp prestained protein standards (Invitrogen, Carlsbad, Calif.). The gel was stained with the EZ Blue staining reagent (Sigma Cat G1041). Gels were scanned on a desk top scanner (HP ScanJet Model G3010) and imaging data analyzed by ImageJ software (NIH).

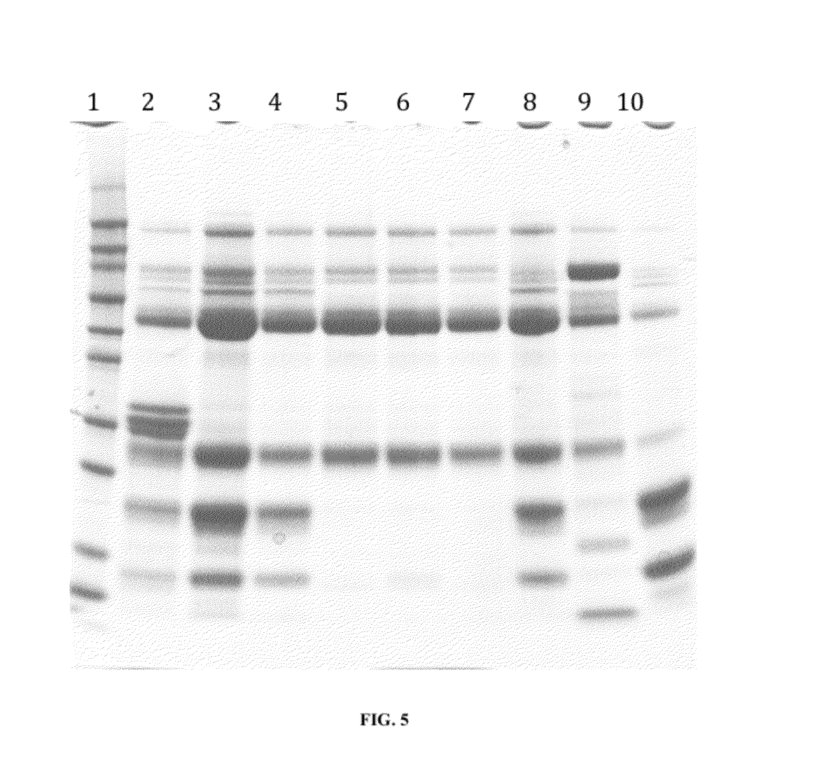

FIG. 5 shows reducing SDS PAGE analysis. All the samples shown in this example are from the process employing diatomaceous earth as a filter aid, except for the sample in Lane 7. Lanes 1-10 were loaded as follows: Lane 1, Molecular weight markers; Lane 2, starting colostrum. Lane 3, Decaseinated whey (load for MEP or Capto-S column), Lane 4, Capto-S Flow Through, Lane 5, MEP eluate, Lane 6, TFF-Concentrated MEP eluate, Lane 7, MEP Eluate (no diatomaceous earth), Lane 8, TFF-concentrated Capto S-Flow through, Lane 9, Capto-S 1 M NaCl strip, Lane 10, Permeate from UF/DF step of CaptoS-TFF process. When analyzed, MEP process and Capto-S-TFF processes produced different profiles, for instance with the MEP process having a preponderance of lactoferrin as a likely contaminant (see Example I below) and the Capto-S process having lactoglobulin as a likely contaminant. The identity of contaminants was determined by comparison to standards run on SDS-PAGE, consideration of isoelectric points, and results from mass spectrometry analysis. Subsequently, appropriate optimization and polishing steps can be applied to achieve different preferred embodiments of polyclonal antibody compositions.

### Example 11

### Quantitation of IgM and IgA in Polyclonal Compositions

Commercially available ELISA kits (Cat.#E11-101 and #E11-121, Bethyl Laboratories, Montgomery, Tex.) were used to determine the levels of IgM and IgA, respectively, in different preparations. Anti-bovine IgM or IgA antibodies are precoated on the 96-well strip plates provided. The plates were washed, blocked, and serial dilutions of samples were added, washed, and binding detected with either horseradish-peroxidase conjugated, affinity purified goat anti-bovine IgM or goat anti-bovine IgA and 3,3′,5,5′-tetramethylbenzidine (TMB) as substrate. Material purified by MEP chromatography was compared with the flow through material from Capto-S chromatography. Data are expressed as mg of the isotype per gram of product based on protein concentration using the BCA assay.

| Sample | IgA (mg/g) | IgM (mg/g) |
|---|---|---|
| Defatted colostrum | 24 | 51 |
| MEP | 108 | 14 |
| Capto-S | 136 | 72 |

IgA levels were increased in both purified preparations, reflecting enrichment of immunoglobulin as impurities (particularly casein) is removed. IgM is slightly enriched in the Capto-S preparation, but is significantly depleted in the MEP preparation. This further demonstrates the superiority of the Capto-S method over MEP. In the Capto-S preparation, 13% of the protein was IgA and 7% was IgM, reflecting retention of all IgA and loss of approximately 50% of the IgM, based on typical levels of these isotypes in colostrum.

### Example 12

### Selective Precipitation to Remove Lactoferrin

Selective precipitation is a technique that can concentrate a protein of interest or remove a contaminating protein. In this experiment, it was found that neutralization of acidified, defatted, decaseinated colostrum with dibasic phosphate selectively precipitated lactoferrin. Defatted colostrum was thawed and heated to 42° C. and diluted with 1.5× volumes of water. The solution was acidified with 5% lactic acid to a final pH of 4.6. Casein was removed by crude filtration followed by continuous flow centrifugation and the acidified material was held overnight at 2-8° C. In the morning, 4 g/L diatomaceous earth was added and the material filtered through a CUNO Zeta Plus Capsule filter. Different neutralization conditions were then compared, varying temperature, rate of neutralization, and use of NaOH or Na(P)dibasic. In all cases, some turbidity was observed and precipitated material was removed by centrifugation and analyzed by reducing SDS PAGE.

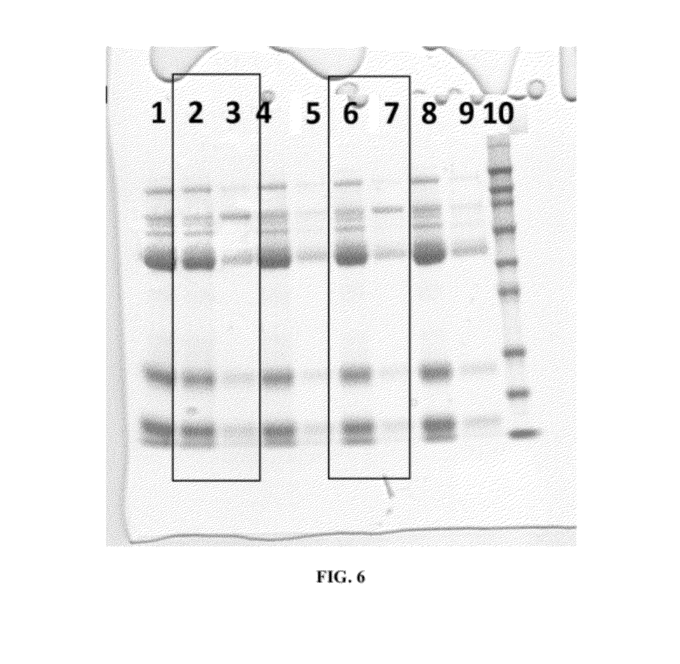

FIG. 6 shows the following; Lane 1, post-casein starting material, Lane 2, Supernatant, neutralized with sodium dibasic phosphate at a rapid rate, Lane 3, pellet fraction, neutralized with sodium dibasic phosphate at a rapid rate, Lane 4, Supernatant fraction, neutralized with sodium hydroxide at a rapid rate, Lane 5, Pellet fraction, neutralized with sodium hydroxide at a rapid rate, Lane 6, Supernatant Fraction, neutralized with sodium phosphate dibasic over 20 min, Lane 7, Pellet Fraction, neutralized with sodium phosphate dibasic over 20 min, Lane 8, Supernatant, NaOH fraction neutralized over 20 min, Lane 9, Pellet fraction, neutralized with sodium hydroxide over 20 min, Lane 10, Molecular Weight marker.

In this experiment, a 75 kDa protein of the same relative mobility of lactoferrin (compared to a commercially available standard) was found enriched in the pellet fraction when sodium phosphate dibasic was used to neutralize the pH in preparation of whey from post-casein colostrum (lanes 2-3, 6-7) compared to sodium hydroxide (lanes 4-5, 8-9). The relative enrichment of putative lactoferrin was accompanied by a white precipitate, likely to be calcium phosphate.

Based on this result, a pilot scale run was carried out using sodium dibasic phosphate as a neutralization agent and using the continuous flow centrifuge to remove the precipitated material. However, the calcium phosphate precipitate proved to be extremely difficult to clean from the processing equipment. Therefore, although this method may be useful at bench scale, it is not a method that is useful at a pilot or production scale.

### Example 13

### Advantage of Sequential Flow Through Strategy-Bench Scale Study

The experiment described here shows bench scale chromatography using resins that reliably scale to pilot and process scales, followed by analysis of the protein profiles using reducing SDS PAGE. Colostral whey was prepared at pilot scale and samples were loaded onto 5 ml columns as indicated below.

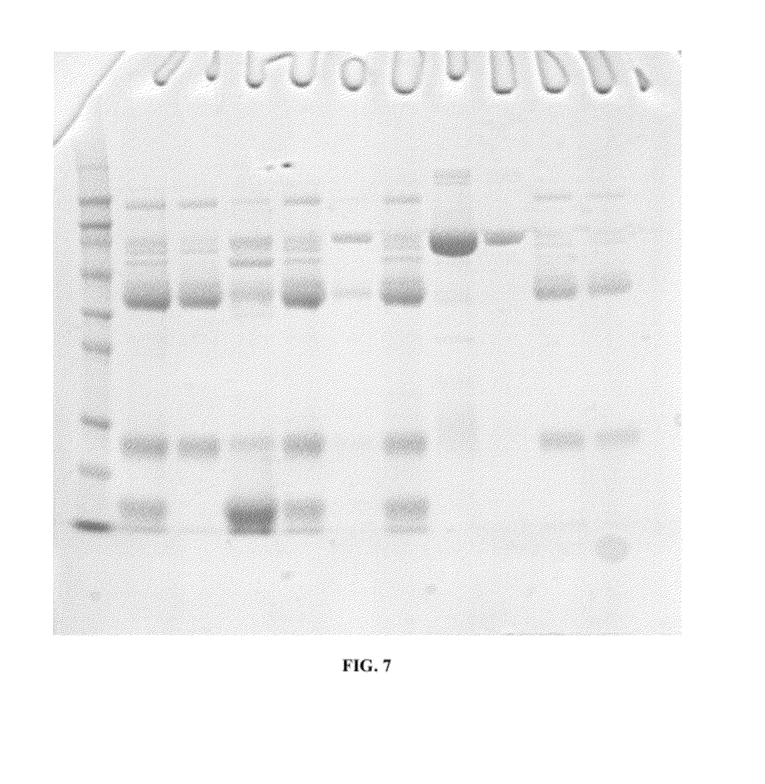

FIG. 7 shows reducing SDS PAGE analysis from this experiment. The lanes are numbered from left to right. Lane 1, Molecular weight markers, Lane 2, starting material for the column resin binding experiments, Lane 3, Capto-Q Flow through fraction, Lane 4, Capto Q 1 M strip, Lane 5, Capto-S Flow through, Lane 6, Capto-S 1 M strip, Lane 7, Capto-S Flow through process sample from a different batch, Lane 8, bovine lactoferrin (5 μg, Bethyl Laboratories), Lane 9, bovine lactoferrin (0.5 μg), Lane 10, Sequential column flow through Capto-S followed by Capto-Q, Lane 11, Serial column flow through Capto-Q followed by Capto-S.

Together with the data in Example 10, this experiment suggests a sequential flow through chromatography process with Capto-S and Capto-Q can result in an improved process when compared with MEP column chromatography. In particular, results with the novel, strategy of flow through Capto-Q in series with flow through Capto-S looks particularly promising.

### Example 14

### Serial Capto-S and Capto-Q Chromatography Scaled to 30 L Colostrum and 3 L Columns

Fat was removed from 30 L of colostrum by continuous flow centrifugation in a Westphalia apparatus (SA-1-02-175, GEA Mechanical Equipment US, Inc., Northvale, N.J.), acid precipitation by lactate addition at 42° C. (DL-Lactic Acid, 85% solution, (Fisher Scientific, Waltham, Mass.) and crude filtration. Following the crude filtration, the material was held overnight at 2-8° C. and then neutralized by Tromethamine addition (Trizma Base, Sigma Aldrich, St Louis Mo.). The neutralized whey was clarified by continuous flow centrifugation. Next, in a flocculation step, diatomaceous earth filter agent (Sigma Aldrich, St Louis, Mo.) was added to 4 g/mL prior to the first filter capsule (Gregory, A. G., U.S. Pat. No. 5,707,678), with stirring for 10 min. The clarification filter train consisted of a 20 μm Alpha fibrous polypropylene (Meissner Filtration Products, Camarillo, Calif.)/0.45 μm polypropylene filter CLMFO.45-222 (Meissner Filtration Products, Camarillo, Calif.)/0.2 μm filter (Pall Corporation, Port Washington, N.Y.).

Capto-S resin (3 L bed volume) and Capto-Q resin (3 L bed volume) were packed in two INdEX 140/500 columns (GE Healthcare Bio-Sciences Corp., Piscataway, N.J.), connected in series. Prior to loading the sample, the columns were washed sequentially with 12 L reverse osmosis water, 12 L 0.5 M NaCl, 12 L reverse osmosis water, 12 L 1 M NaCl, 12 L reverse osmosis water, then 60 L 1 M Tris-HCl pH 6.8. The whey (30 L) was pumped onto the column at a flow rate of 0.5 L/min, and the column was washed with 2.5 column volumes of equilibration buffer. Absorbance at 280 nm was monitored using an inline flow cell (PendoTECH, Princeton, N.J.). Collection of flow through was stopped when A280 approached baseline levels. After chromatography, the product was concentrated by ultrafiltraton (50 kDa NMWCO filter), using a Pall Pharmaceutical Series apparatus, Pall Corporation, Port Washington, N.Y.) then diafiltered versus 5 volumes of reverse osmosis water. The product was concentrated to >75 mg/mL by ultrafiltration. Terminal heat treatment was performed at 60° C. for 10 hours.

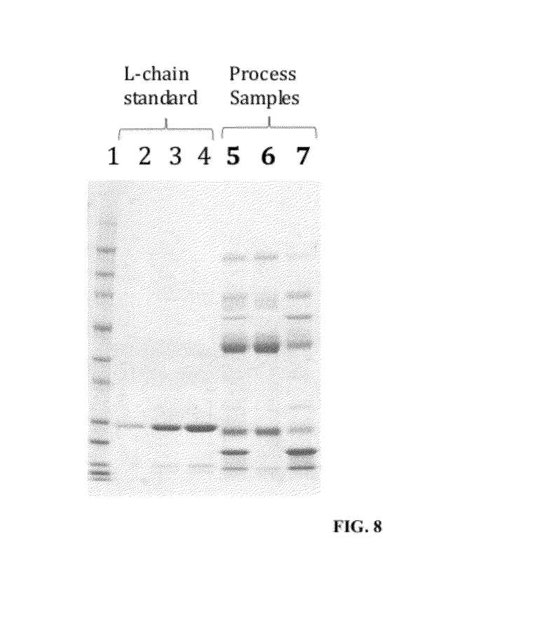

FIG. 8 shows the reducing SDS PAGE analysis results from this 30 L pilot scale column chromatography on Capto-S and Capto-Q, connected in series. Proteins bound to Capto-S and Capto-Q columns were assessed by stripping the column with 1 M NaCl. Lane 1, Protein Molecular Weight Markers, Lanes 2-4, increasing loads of IgG L-chain standard used as a control to quantify immunoglobulin content (electrophoresis, >99% pure, from human myeloma plasma, obtained from Sigma Aldrich, St Louis, Mo.) Lane 5, load prior to serial chromatography, Lane 6, Flow through from Capto-S/3 L Capto-Q serial columns, Lane 7, Eluate of Serial Columns (1M NaCl). Gels were stained with Coomassie Brilliant Blue and electronically imaged using a MFC-9120CN scanner (Brother). ImageJ 1.45s software (National Institutes of Health, Bethesda, Md., imagej.nih.hov/ij/docs) was used create densitometry plots. Peak area was measured by integrating the baseline-subtracted area between the half-peak heights.

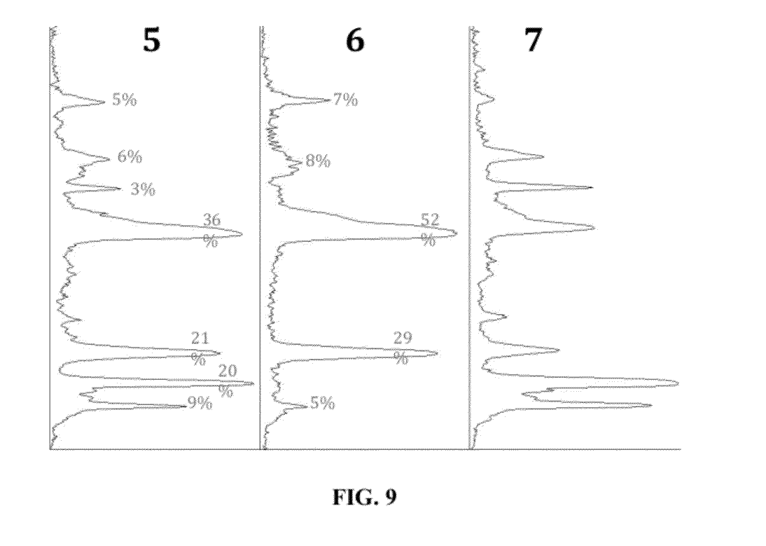

FIG. 9 shows densitometry traces of lanes 5, 6 and 7. Comparison of lanes 5 and 6 shows diminution of non-Ig proteins and concentration of Ig proteins, heavy and light chains. In the composition shown in lane 6, 81% of the product is present in immuglobulin heavy and light chains. The high molecular weight band is aggregated Ig heavy chain (see Example 14) and the majority of the material present in the 70-80 kDa section is also product-related (IgM and secretory component—see Example 14). Therefore 95% of the product is immunoglobulin.

The trace of Lane 7 shows that a number non-Ig proteins preferably bind to the resins. Taken together with the traces from Lanes 5 and 6 and other data, it was concluded that serial flow through chromatography is a powerful method for preparation of polyclonal antibody compositions from colostrum. The identities of proteins in the flow-through and eluate were investigated further in the examples below. It will be readily recognized that this process or variations thereof will provide the appropriate yields of polyclonal antibody compositions suitable for oral administration.

### Example 15

### Serial Capto-S and Capto-Q Chromatography Scaled to 80 L Colostrum (Prophetic)

Having exemplified the method for preparing antibody compositions at 30 L scale, it will be recognized by those skilled in the art that the procedure can be scaled up to 80 L without extensive experimentation. Preparation of antibody compositions from 80 L of colostrum will be carried out as follows as described below.

Fat is removed from colostrum (80 L) by continuous flow centrifugation in a Westphalia apparatus (SA-1-02-175, GEA Mechanical Equipment US, Inc., Northvale, N.J.). The resulting defatted colostrum is diluted with 2 volumes of reverse osmosis water, and lactic acid is added to a final pH of 4.6 at 42° C. (DL-Lactic Acid, 85% solution, Fisher Scientific, Waltham, Mass.) to precipitate casein, with mixing by broad blade vertical impeller or equivalent mixing apparatus. Following the crude filtration or equivalent step such as cheese press to remove casein, the material is held overnight at 2-8° C. and then neutralized by Tromethamine addition (Trizma Base, Sigma Aldrich, St. Louis Mo.). Diatomaceous earth filter agent (Sigma Aldrich, St Louis, Mo.) is added to 4 g/mL prior to the first filter capsule with stirring for 10 min. The clarification filter train consists of a 20 μm Alpha fibrous polypropylene (Meissner Filtration Products, Camarillo, Calif.)/0.45 μm polypropylene filter CLMFO.45-222 (Meissner Filtration Products, Camarillo, Calif.)/0.2 μm filter (Pall Corporation, Port Washington, N.Y.). It will be recognized that other filter trains from these or other manufacturers will also equivalently prepare the sample for chromatography.

In this example, scale up is accomplished by dividing the sample into three aliquots and subjecting each portion to serial chromatography, with washing of the column set up in between samples. Capto-S resin (3 L bed volume) and Capto-Q resin (3 L bed volume) is packed in two INdEX 140/500 columns (GE Healthcare Bio-Sciences Corp., Piscataway, N.J.), connected in series. Prior to each sample load, the serial column set up is washed with 12 L reverse osmosis water, 12 L 0.5 M NaCl, 12 L reverse osmosis water, 12 L 1 M NaCl, 12 L reverse osmosis water, then 1 M Tris-HCl pH 6.8 (until pH is stabilized at 6.8). After the wash steps, the column is equilibrated with 18 L 10 mM Tris-HCl, pH 6.8. The pH and conductivity of the whey is measured and the whey is pumped onto the columns at a flow rate of 0.5 L/min, and the column set up is washed with 2.5 column volumes of equilibration buffer. Absorbance at 280 nm and pH will be monitored using an inline flow cell (PendoTECH, Princeton, N.J.). Collection of flow through is stopped when A280 approaches baseline levels. After chromatography, the pH and conductivity is measured and the pH is found to be within 0.2 pH units of the pH of the load material and the conductivity is found to be within 1 milliSiemens/cm of the load material. The product is concentrated by ultrafiltration (50 kDa NMWCO filter), using a Pall Pharmaceutical Series apparatus, (Pall Corporation, Port Washington, N.Y.) then diafiltered versus 5 volumes of reverse osmosis water. The product is concentrated to >75 mg/mL by ultrafiltration. Terminal heat treatment is performed at 60° C. for 10 hours.

### Example 16

### Investigation by Mass Spectrometry of the Identity of Proteins in the Polyclonal Antibody Preparation

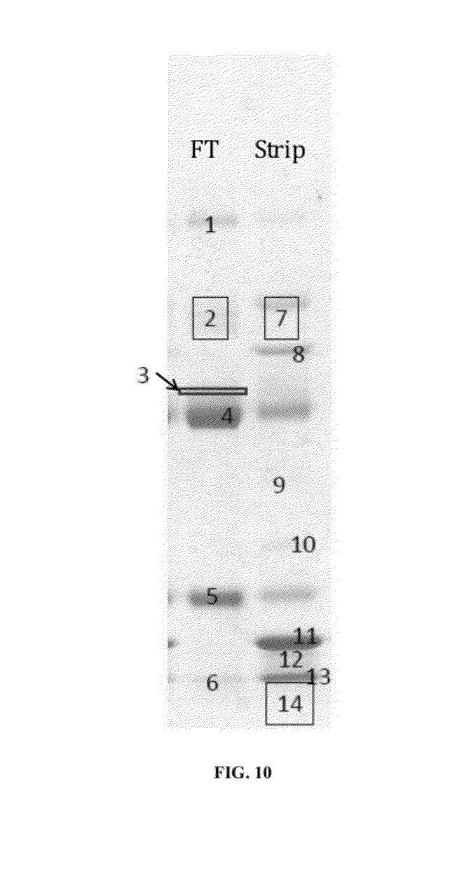

52 kg of colostral whey was loaded on to two 3 L columns of Capto-S and Capto-Q in series as described in Example 14. The flow through and strip fractions were analyzed by reducing SDS-PAGE and FIG. 10 shows the identity of bands excised from the gel. Samples were subjected to mass spectrometry. The analysis was performed on an LTQ-Orbitrap apparatus (Fisher ThermoScientific, Waltham, Mass.) at the University of Massachusetts. The resulting peptide sequences were used to search the NCBI nr database.

| Band | | |
|---|---|---|
| number | Sample | Most prevalent sequences |
| 1 | Flow-through | IgG1 heavy chain |
| 2 | Flow-through | Transferrin, IgM, secretory component |
| | | (poly IgR) |
| 3 | Flow-through | IgG1 |
| 4 | Flow-through | IgG1 |
| 5 | Flow-through | Ig light chain (primarily lambda, some kappa) |
| 6 | Flow-through | Alpha-lactalbumin, keratin |
| 7 | Strip | Lactoferrin, transferrin, IgM, some |
| | | lactoperoxidase |
| 8 | Strip | Bovine serum albumin |
| 9 | Strip | Zinc alpha 2 glycoprotein, complement C3 |
| 10 | Strip | IgG1 (presumably fragments) |
| 11 | Strip | Beta-lactoglobulin |
| 12 | Strip | Pancreatic ribonuclease |
| 13 | Strip | Alpha-lactalbumin |
| 14 | Strip | Ig heavy chain, keratin |

An analysis of the flow-through material confirmed that the major bands on reducing SDS PAGE (bands 4 and 5) represent IgG heavy and light chains. The smearing above band 4 (band 3) is again IgG heavy chain and presumably represents different glycoforms. The high molecular weight band (band 1) seen in all analyses of bovine immunoglobulin is an aggregate of IgG heavy chain. A triplet of bands is seen in the sample labeled band 2. This triplet consists primarily of secretory component (79 kDa), IgM (76 kDa) and transferrin (73 kDa). Both secretory component and IgM are desired components of the composition, while transferrin is an impurity. The remaining low molecular weight band includes the impurities alpha-lactalbumin and keratin. These impurities will be removed during downstream polishing on ultrafiltration diafiltration.

An analysis of the material stripped from the columns confirmed that the process removed lactoferrin, bovine serum albumin, beta-lactoglobulin, and alpha-lactalbumin, as well as some immunoglobulin and some minor impurities.

This analysis showed that extraneous proteins that may confound production of a pharmacologically active polyclonal antibody preparation can be removed using this strategy, and that further polishing steps can be applied to produce compositions suitable for patient populations including those with compromised gastrointestinal systems.

### Example 17

### Direct Comparison of Compositions Purified Using Different Methods

A direct comparison was made of compositions of colostrum purified using four different methods: thioester T-gel chromatography (Example 2), ammonium sulfate precipitation (Example 4), MEP chromatography (Example 8) and Capto-S/Capto-Q serial chromatograph (Example 14). Samples of each preparation were analyzed by reducing SDS PAGE and by ELISA to quantify the levels of lactoferrin, alpha-lactalbumin, beta-lactoglobulin. Samples were also assayed by ELISA to quantify the levels of lactoperoxidase and IGF-1.

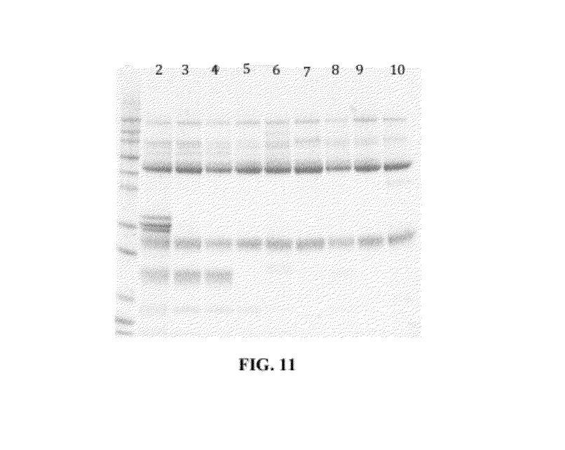

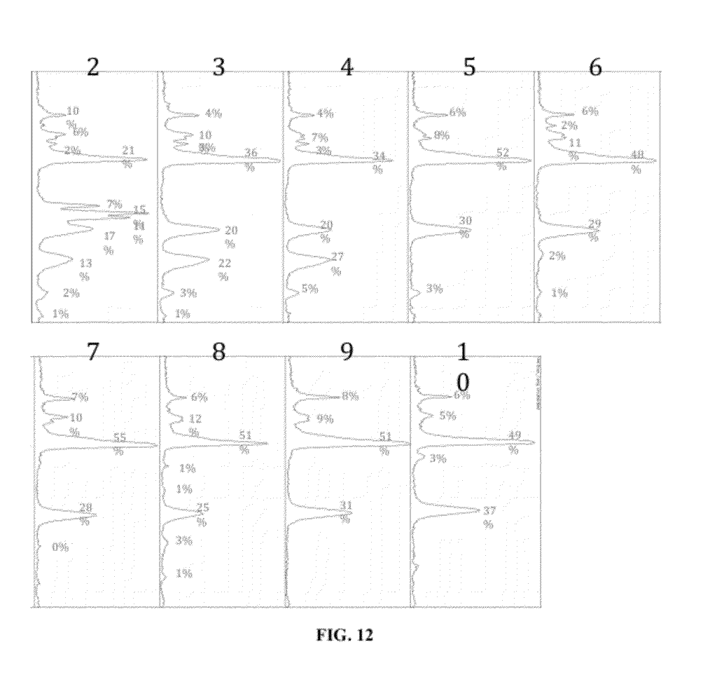

FIG. 11 shows a reducing SDS PAGE analysis of these different compositions. Lane 1: molecular weight markers; Lane 2: defatted pooled colostrum; Lane 3: Defatted, decaseinated whey; Lane 4, flow through Capto-S only; Lane 5, Flow through Capto-Q only; Lane 6, Flow through Capto-S/Capto-Q; Lane 7, MEP chromatography; Lane 8, ammonium sulfate-purified antibody preparation; Lane 9, T-gel-purified antibody preparation; Lane 10, affinity purified antibody specific for murine TNF. FIG. 12 shows a densitometric analysis of this gel. These data confirm the results presented in the examples above and show that the four methods under investigation result in roughly comparable levels of purity as judged by this method (note that the T-gel and ammonium sulfate products were purified at the bench scale while the MEP and Capto-S/Capto-Q products were produced at pilot scale).

More significant differences were seen when assays were performed to quantify levels of specific impurities.

The samples were analyzed in the BCA assay to quantify total protein and by ELISA to quantify the levels of specific impurities. A commercially available ELISA kit (Cat. #E10-126, Bethyl Laboratories, Montgomery, Tex.) was used to quantify lactoferrin. Per manufacturer's recommendation, ELISA plates were coated with a 1:100 dilution of goat-anti bovine lactoferrin coating antibody reagent provided. The plates were washed, blocked, and serial dilutions of samples were added, washed, and binding detected with horseradish-peroxidase conjugated, affinity purified goat anti-bovine lactoferrin and 3,3′,5,5′-tetramethylbenzidine (TMB) as substrate. Commercially available ELISA kits (Cat. #E10-125 and #E10-128, Bethyl Laboratories, Montgomery, Tex.) were used to quantify beta-lactoglobulin and alpha-lactalbumin, respectively. Per the manufacturer's recommendation, ELISA plates were coated with a 1:100 dilution of the goat-anti bovine beta-lactoglobulin or alpha-lactalbumin coating antibody reagent provided. The plates were washed, blocked, and serial dilutions of samples were added, washed, and binding detected with horseradish-peroxidase conjugated, affinity purified goat anti-bovine beta-lactoglobulin or alpha-lactalbumin, respectively and 3,3′,5,5′-tetramethylbenzidine (TMB) as substrate. Commercially available ELISA kits (Cat.#KT-20283 and #KT-18278, Kamiya Biomedical Co., Seattle, Wash.) were used to determine the levels of bovine lactoperoxidase (LPO) and insulin-like growth factor I (IGF-I), respectively, in different preparations. Anti-bovine LPO or IGF-I antibodies are precoated on the 96-well strip plates provided. Serial dilutions of samples and calibrator standards were added and incubated prior to addition of detection reagent A. After additional incubation, wells were washed and detection reagent B added and incubated. Finally, wells were washed and incubated with 3,3′,5,5′-tetramethylbenzidine (TMB) substrate solution, followed by stop solution prior to being read at 450 nm.

| | | | Beta- |
|---|---|---|---|
| | Lactoferrin | Alpha-lactalbumin | lactoglobulin |
| Sample | mg/g | mg/g | mg/g |
| Defatted colostrum | 10 | 88 | 197 |
| Whey | 11 | 146 | 70 |
| Capto-S/Capto-Q | 0.3 | 75 | 0.5 |
| MEP | 30 | 4.0 | 7.6 |
| Ammonium sulfate | 2.4 | >20 | 25 |
| T-gel | 0.5 | ND | ND |

| Sample | IGF-1 mg/g | Lactoperoxidase mg/g |
|---|---|---|
| Whey | >5.1 | 64 |
| Capto-S/Capto-Q 8Feb | 0.09 | 0.18 |
| Capto-S/Capto-Q 10Feb | 0.05 | 0.21 |
| MEP | 41 | 21 |
| T-gel | 0.12 | 1.8 |

### Example 18

### Purification of the Antigen-Specific Component of AVX-176 by Affinity Chromatography

Six Holstein cows were immunized during their last trimester of pregnancy with three injections of gliadin and adjuvant. Colostrum was collected for the first four days after calving. Colostrum samples from all six gliadin-immunized cows were pooled. Fat was removed by centrifugation and casein was precipitated by acidification to pH 4.6. Anti-gliadin antibody (AVX-176) was purified using thiophilic adsorbent chromatography as described in Example 2. A 33-mer peptide that is known to be one of the immunodominant peptides in gliadin was synthesized; the peptide is called 56-89 and the sequence is LQLQPFPQPQLPYPQPQLPYPQPQLPYPQPQPF (SEQ ID NO: 1). An affinity column was prepared by linking 20 mg 56-89 to 12.5 ml NHS-Sepharose. The gliadin-specific component of the antibody was purified by loading 210 mg of the T-gel-purified preparation onto the column, washing first with PBS and then with 250 mM NaCl in PBS, and eluting the specific antibody with 0.1 M glycine pH 2.7. Fractions were collected into Tris buffer to immediately neutralize the solution.

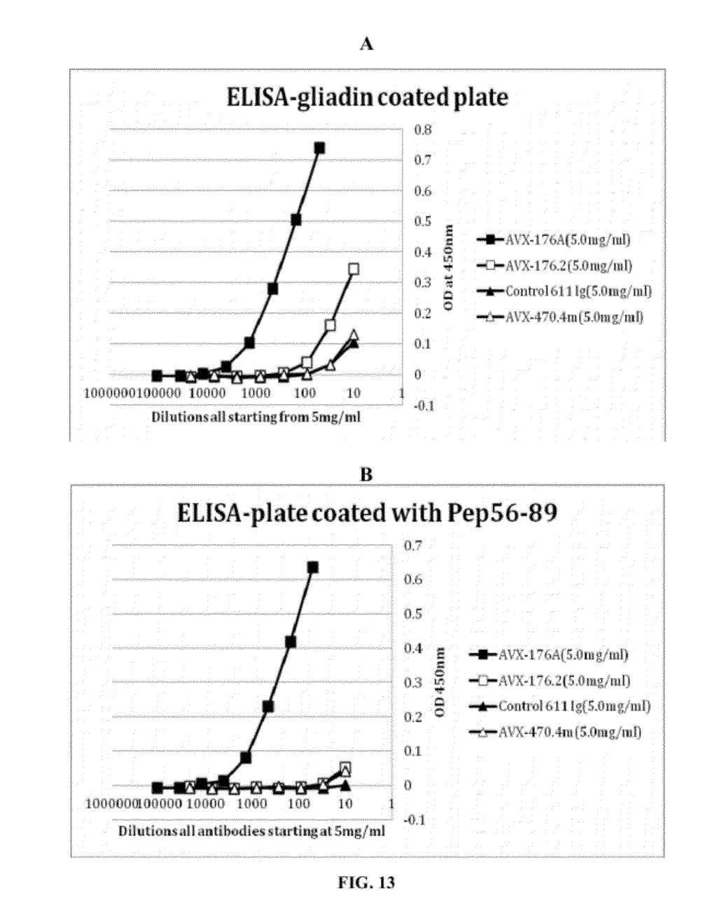

The activity of AVX-176 and the affinity-purified component (AVX-176A) were measured by ELISA. ELISA plates were coated with gliadin dissolved in urea (3 mol/L) and carbonate-bicarbonate coating buffer (100 mM) or with peptide 56-89 at 20 mg/ml in carbonate-bicarbonate coating buffer (100 mM). Serial dilutions of AVX-176A, AVX-176 or control antibodies AVX-470m specific for murine TNF or AVX-610 control bovine immunoglobulin, were added to the plates and binding was assessed using standard techniques. The results are shown in FIG. 13.

This example demonstrates that the antigen-specific component of a polyclonal antibody composition can be enriched by chromatography on an antigen affinity column. Through this enrichment process, the non-specific antibodies of the composition have been depleted.

### Example 19

### Reducing SDS PAGE Analysis of Composition Purified on Capto-S/Capto-Q Chromatography Followed by Ultrafiltration

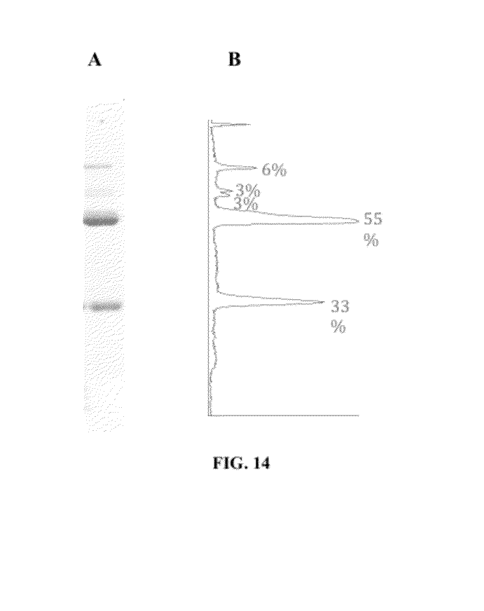

Immunoglobulin was purified from colostral whey as described in Example 14. The material that flowed through the serial Capto-S and Capto-Q columns was subjected to ultrafiltration on a 30,000 molecular weight cut-off membrane and the retentate was analyzed by reducing SDS-PAGE. FIG. 14 shows the gel from the SDS-PAGE analysis (A) and the densitometric analysis of the gel (B).

A comparison of the gel in this example with that in FIGS. 11 and 12 demonstrates that the addition of the ultrafiltration step cleanly removes the alpha-lactalbumin remaining in the Capto-S/Capto-Q flow through. The material analyzed in FIGS. 11 and 12 had to a peak area of alpha-lactalbumin of 1% in the densitometry analysis which corresponded to a concentration of 75 mg/g of alpha-lactalbumin by ELISA (see Example 17). Following ultrafiltration, there was no alpha-lactalbumin detectable on the SDS-PAGE analysis, indicating that the level of alpha-lactalbumin is <15 mg/g.

Based on densitometry, this composition is 97% immunoglobulin: 55% Ig heavy chain (IgG and IgA), 33% Ig light chain (kappa and lambda), 3% secretory IgM heavy chain and an impurity of 3% transferrin.

Capto-Q is a strong anion exchanger and Capto-S is a strong cation exchanger. Typically one would optimize the pH to bind one resin or the other, based on the pI of the protein. However, polyclonal antibodies have a broad pI range, complicating this approach. A novel approach to using these columns such that the highest yield of purified and isolated immunoglobulin could be achieved, was to choose a pH in the middle of the predicted pI range for the polyclonal immunoglobulin, such as a pH in the range of 6.6 to 7.0.

| | pI | pH 7 | Should | pH 5.8 | Should |
|---|---|---|---|---|---|
| Protein | value | charge | Bind | charge | Bind |
| b-lactoglobulin | 5.2-5.4 | negative | Q (+) | mostly | nothing |
| | | | | neutral | |
| a-lactalbumin | 4.3-5.1 | negative | Q (+) | neutral to | weakly |
| | | | | negative | to Q (+) |
| polyclonal bovine | 5.8-7.3 | neutral to | weakly to | neutral to | weakly |
| immunoglobulin | | positive | S (−) | negative | to Q (+) |
| population | | | | | |
| lactoferrin | 7.8-8.0 | weakly | weakly to | positive | S (−) |
| | | positive | S (−) | | |
| lactoperoxidase | 9.2-9.9 | positive | S (−) | strongly | S (−) |
| | | | | positive | |
| BSA* | 5.13 | negative | Q (+) | mostly | nothing |
| | | | | neutral | |

The experiments described herein determined that it was preferable to use a flow-through approach rather than bind and elute as the flow through provides faster throughput, less use of expensive buffers, and resulted in a more highly purified preparation. If the conditions are not correct, then some immunoglobulin will bind to the resin, resulting in reduced yields. The novel approach described herein optimized the conditions that resulted in the highest yield with the highest purity of immunoglobulin composition

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

What is claimed is:

1. A composition consisting essentially of isolated and purified immunoglobulin derived from the colostrum of a bovine that has been immunized with all or a portion of a target antigen, wherein composition is at least 90% immunoglobulin as determined by reducing SDS-PAGE/densitometry and contains less than about 1 mg of lactoferrin per gram of total protein present in the composition wherein the total protein content of the composition is measured by bicinchonic acid (BCA) assay and the level of lactoferrin is measured by ELISA, wherein the composition binds the target antigen in an in-vitro antibody binding assay.

2. The composition of claim 1, wherein the composition contains less than 0.3 mg/g lactoferrin.

3. The composition of claim 1, wherein the composition contains less than 20 mg/g of alpha-lactalbumin.

4. The composition of claim 1, wherein the composition contains less than 5 mg/g of beta-lactoglobulin.

5. The composition of claim 1, wherein the composition contains less than 2 mg/g of lactoperoxidase.

6. The composition of claim 1, wherein the composition contains less than 1 mg/g of insulin-like growth factor-1 (IGF-1).

7. The composition of claim 1, wherein the composition is at least 95% immunoglobulin as determined by reducing SDS-PAGE.

8. A composition consisting essentially of isolated and purified immunoglobulin derived from the colostrum of a bovine that has been immunized with all or a portion of a target antigen, wherein composition is at least 90% immunoglobulin as determined by reducing SDS-PAGE/densitometry and contains less than about 10 mg of lactoferrin per gram of total protein present in the composition wherein the total protein content of the composition is measured by bicinchonic acid (BCA) assay and the level of lactoferrin is measured by ELISA, wherein the composition binds the target antigen in an in-vitro antibody binding assay and wherein the preparation of the composition comprises the steps of:
(a) filtering the whey derived from the colostrum of a bovine immunized with a target antigen through an anion exchange column or a cationic exchange column;
(b) collecting the flow through of the column in step (a); and
(c) concentrating the flow through of step (b) by ultrafiltration.

9. A composition consisting essentially of isolated and purified immunoglobulin derived from the colostrum of a bovine that has been immunized with all or a portion of a target antigen, wherein composition is at least 90% immunoglobulin as determined by reducing SDS-PAGE/densitometry and contains less than about 10 mg of lactoferrin per gram of total protein present in the composition wherein the total protein content of the composition is measured by bicinchonic acid (BCA) assay and the level of lactoferrin is measured by ELISA, wherein the composition binds the target antigen in an in-vitro antibody binding assay, and wherein the preparation of the composition comprises the steps of:
(a) adjusting the pH of whey derived from the colostrum of a bovine immunized with a target antigen to a pH of 6.6 to 7.0;
(b) filtering the whey through an anion exchange column connected in series with a cation exchange column wherein the whey sequentially flows through both columns connected in series without addition of materials that change the salt concentration or pH;
(c) collecting the flow through after it passes through both columns of step (b) without addition of materials that change the salt concentration or pH before collection occurs; and
(d) concentrating the flow through of step (b) by ultrafiltration.

10. A pharmaceutical formulation consisting essentially of an optional, pharmaceutically acceptable excipient and a composition consisting essentially of isolated and purified immunoglobulin derived from the colostrum of a bovine that has been immunized with all or a portion of a target antigen, wherein composition is at least 90% immunoglobulin as determined by reducing SDS-PAGE/densitometry and contains less than about 10 mg of lactoferrin per gram of total protein present in the composition wherein the total protein content of the composition is measured by bicinchonic acid (BCA) assay and the level of lactoferrin is measured by ELISA, wherein the composition binds the target antigen in an in-vitro antibody binding assay.

11. The pharmaceutical formulation of claim 10 formulated for topical delivery to the oral cavity, oral delivery, or rectal delivery.

12. The pharmaceutical formulation of claim 10, wherein the composition is in the form of a spray dried powder or lyophilized powder.

13. The pharmaceutical formulation of claim 10, wherein the composition is at least 95% immunoglobulin as determined by reducing SDS-PAGE/densitometry.

14. A process for producing a composition consisting essentially of isolated and purified immunoglobulin derived from the colostrum of a bovine that has been immunized with all or a portion of a target antigen, wherein composition is at least 90% immunoglobulin as determined by reducing SDS-PAGE/densitometry and contains less than about 10 mg of lactoferrin per gram of total protein present in the composition wherein the total protein content of the composition is measured by bicinchonic acid (BCA) assay and the level of lactoferrin is measured by ELISA, wherein the composition binds the target antigen in an in-vitro antibody binding assay comprising the steps of:
(a) adjusting the pH of whey derived from the colostrum of a bovine immunized with a target antigen to a pH of 6.6 to 7.0;
(b) filtering the whey through an anion exchange column connected in series with a cation exchange column wherein the whey sequentially flows through both columns connected in series without addition of materials that change the salt concentration or pH;
(c) collecting the flow through after it passes through both columns of step (b) without addition of materials that change the salt concentration or pH before collection occurs; and
(d) concentrating the flow through of step (b) by ultrafiltration.

15. The process of claim 14, wherein the composition is at least 95% immunoglobulin as determined by reducing SDS-PAGE/densitometry.

16. The process of claim 14, further comprising lyophilizing or spray-drying the concentrated material of step (d).

17. The composition of claim 1, wherein immunoglobulins present in the composition are immunoglobulins of more than one isotype.

18. The composition of claim 17, wherein the immunoglobulins present in the composition comprise the isotypes IgG, IgM and IgA.

19. The composition of claim 18, wherein IgM and IgA together comprise at least 10% of the total immunoglobulins present in the composition as measured by ELISA.

20. The method of claim 14, wherein the conductivity of the whey in step (a) is the same as the conductivity of the flow through of step (c).

21. The method of claim 14, wherein the pH of the flow through of step (c) is the same as the pH of the whey in step (a).

22. The composition of claim 1, wherein the target antigen is TNF.

23. A method of treating inflammatory bowel disease (“IBD”), oral mucositis or intestinal mucositis comprising administering to a patient a therapeutically effective amount of the composition of claim 22.

24. The method of claim 23, wherein the patient is a human patient and the composition comprises polyclonal antibodies specific to human TNF.

25. A composition consisting essentially of isolated and purified polyclonal antibodies derived from the colostrum of a bovine that has been immunized with all or a portion of a target antigen, wherein non-immunoglobulin factors are depleted from the composition to a level that is below 10 fold the normal levels contained in colostrum and the composition comprises polyclonal antibodies specific for a target antigen.

26. The composition of claim 1 wherein the in vitro antibody binding assay is an ELISA.

27. The method of claim 8 wherein the in vitro antibody binding assay is an ELISA.
